SEQUENCE LISTING (11 sequences)

SEQ ID NO: 1 - protein, 94 aa, Artificial Sequence
  RKIP motif consensus amino acid sequence
TLXXXDPDXPXXXXXXXXEXXHXXXXXXXXXXXXXXXXXXXXXXXXXXXXXXXXXXXXXXXXXXXXXXXXXXYXXXXPXXXXGXHRXVXXXXXQ

SEQ ID NO: 2 - protein, 187 aa, Homo sapiens
MPVDLSKWSGPLSLQEVDEQPQHPLHVTYAGAAVDELGKVLTPTQVKNRPTSISWDGLDSGKLYTLVLTDPDAPSRKDPKYREWHHFLVVNMKGNDISSGTVLSDYVGSGPPKGTGLHRYVWLVYEQDRPLKCDEPILSNRSGKHRGKFKVASFRKKYELRAPVAGTCYQAEWKKYVPKLYEQLSGK

SEQ ID NO: 3 - protein, 187 aa, Mus musculus
  MISC_FEATURE (150)..(150): Xaa = any amino acid
MAADISQWAGPLCLQEVDEPPQHALRVDYAGVTVDELGKVLTPTQVMNRPSSISWDGLDPGKLYTLVLTDPDAPSRKDPKFREWHHFLVVNMKGNDISSGTVLSDYVGSGPPSGTSIHRYVWLVYEQEQPLSCDEPILSNKSGDNRGKFXVETFRKKYNLGAPVAGTCYQAEWDDYVPKLYEQLSGK

SEQ ID NO: 4 - protein, 187 aa, Drosophila
MSDSTVCFSKHKIVPDILKTCPATLLTVTYGGGQVVDVGGELTPTQVQSQPKVKWDADPNAFYTLLLTDPDAPSRKEPKFREWHHWLVVNIPGNQVENGVVLTEYVGAGPPQGTGLHRYVFIVFKQPQKLTCNEPKIPKTSGDKRANFSTSKFMSKYKLGDPIAGNFFQAQWDDYVPKLYKQLSGKK

SEQ ID NO: 5 - protein, 220 aa, C. elegans
MVVLVTRSLLPALFFASRAPFAAATTSARFQRGLATMAAEAFTKHEVIPDVLASNPPSKVVSVKFNSGVEANLGNVLTPTQVKDTPEVKWDAEPGALYTLTKTDPDAPSRKEPTYREWHHWLVVNIPGNDIAKGDTLSEYIGAGPPKTGLHRYVYLIYKQSGRIEDAEHGRLTNTSGDKRGGWKAADFVAKHKLGAPVFGNLFQAEYDDYVPILNKQLGA

SEQ ID NO: 6 - protein, 181 aa, Antirrhinum-CEN
MAAKVSSDPLVIGRVIGDVVDHFTSTVKMSVIYNSNNSIKHVYNGHELFPSAVTSTPRVEVHGGDMRSFFTLIMTDPDVPGPSDPYLREHLHWIVTDIPGTTDSSFGKEVVSYEMPRPNIGIHRFVFLLFKQKKRGQAMLSPPVVCRDGFNTRKFTQENELGLPVAAVFFNCQRETAARRR

SEQ ID NO: 7 - protein, 176 aa, Aradopsis-TFL1
MENMGTRVIEPLIMGRVVGDVLDFFTPTTKMNVSYNKKQVNGHELFPSSVSSKPRVEIHGGDLRSFFTLVMIDPDVPGPSDPFLKEHLHWIVTNIPGTTDATFGKEVVSYELPRPSIGIHRFVFVLFRQKQRRVIFPNIPSRDHFNTRKFAVEYDLGLPVAAVFFNAQRETAARKR

SEQ ID NO: 8 - protein, 219 aa, Yeast
MNQAIDFAQASIDSYKKHGILEDVIHDTSFQPSGILAVEYSSSAPVAMGNTLPTEKARSKPQFQFTFNKQMQKSVPQANAYVPQDDDLFTLVMTDPDAPSKTDHKWSEFCHLVECDLKLLNEATHETSGATEFFASEFNTKGSNTLIEYMGPAPPKGSGPHRYVFLLYKQPKGVDSSKFSKIKDRPNWGYGTPATGVGKWAKENNLQLVASNFFYAETK

SEQ ID NO: 9 - DNA, 189 bp, Homo sapiens
accttggtcctgacagacccggatgctcccagcaggaaggatcccaaatacagagaatggcatcatttcctggtggtcaacatgaagggcaatgacatcagcagtggcacagtcctctccgattatgtgggctcggggcctcccaagggcacaggcctgcaccgctatgtctggctggtttacgagcag

SEQ ID NO: 10 - DNA, 7 bp, Artificial Sequence
  Consensus AP-1 Binding Site Sequence
tgantca

SEQ ID NO: 11 - DNA, 11 bp, Artificial Sequence
  nf-kappa-b binding element consensus sequence
ggggactttcc

(19) United States
(12) Patent Application Publication
(10) Pub. No.: US 2004/0180341 A1
(43) Pub. Date: Sep. 16, 2004

(54) Transcriptional regulation of kinase inhibitors

(75) Inventors: John M. Sedivy, Barrington, RI (US); Walter Kolch, Glasgow (GB); Kam Chi Yeung, Perrysbury, RI (US)

Correspondence Address: PALMER & DODGE, LLP, KATHLEEN M. WILLIAMS, 111 HUNTINGTON AVENUE, BOSTON, MA, 02199, US

(73) Assignee: Brown University

(21) Appl. No.: 10/384,435

(22) Filed: Mar. 7, 2003

(51) Int. Cl. C12Q 1/68; G01N 33/574

(52) U.S. Cl. 435/6; 435/7.23

(57) ABSTRACT

The invention relates to the transcriptional regulation of a family of proteins defined by the presence of an RKIP motif. Proteins comprising the RKIP motif modulate kinases involved in signal transduction pathways. Transcriptional regulation of proteins containing an RKIP motif forms the basis for screening assays for the identification of agents useful for modulating signal transduction pathways subject to RKIP family mediated regulation, and for the diagnosis and treatment of disorders involving inappropriate activities of pathways subject to RKIP family mediated regulation.

### FIELD OF INVENTION

The present invention relates, in general, to identification of therapeutic agents that regulate the transcription of a novel class of protein kinase inhibitors, and to their use in modulating cell proliferation and growth.

### BACKGROUND OF THE INVENTION

Raf-1 initiates the mitogen-activated protein (MAP) kinase cascade. RAF-1 phosphorylates and activates MEK-1, a kinase that activates the extracellular signal regulated kinases, ERKs. This kinase cascade governs the proliferation and differentiation of different cell types (Ferrel Jr. Curr. Top. Dev. Biol. 33:1-60, 1996; Morrison and Cutler, Curr. Opin. Cell Biol. 9:174-179, 1997).

The Raf kinase inhibitor protein (RKIP) is a member of a novel class of protein kinase inhibitors that contain an evolutionarily conserved motif termed the RKIP motif (see U.S. Ser. No. 09/654,281, filed Sep. 1, 2000). RKIP is a negative regulator of the mitogen-activated protein (MAP) kinase cascade initiated by RAF-1 (Yeung et al., 1999, Nature, 401:173-177) and also acts to inhibit NF-κB activation via interaction with NK-κB-inducing kinase (NIK) and transforming growth factor beta-activated kinase 1 (TAK-1) (Yeung et al. Molecular and Cellular Biology 21: 7207-7217, 2001). In addition RKIP interacts with the IKKs; IκB Kinase alpha (IKKα) and IκB Kinase beta (IKKβ) (Yeung et al. Molecular and Cellular Biology 21: 7207-7217, 2001).

### SUMMARY OF THE INVENTION

The present invention relates, in general, to agents that regulate the transcription of a class of protein kinase inhibitors that contain an RKIP motif, and to the use of such agents in modulating signal transduction pathways involving kinases responsive to those inhibitors. The invention also relates to the diagnosis of disorders or diseases related to or associated with inappropriate expression of this class of protein kinase inhibitors.

The invention encompasses a method of identifying an agent that modulates transcription of a DNA sequence encoding a polypeptide that comprises an RKIP-motif comprising the steps of: i) providing a DNA sequence encoding a polypeptide that comprises an RKIP-motif; and (ii) contacting the DNA sequence with a candidate agent; and (iii) measuring the amount of RNA transcribed from the DNA sequence wherein an increase or decrease in the amount of RNA transcribed from the DNA sequence is indicative that the agent is a transcriptional modulator.

The invention encompasses a method of identifying an agent that modulates a signal transduction pathway, the method comprising the steps of: (i) providing a DNA sequence encoding a polypeptide that comprises an RKIP-motif; (ii) contacting said DNA sequence with a candidate agent, and, (iii) measuring the amount of RNA transcribed from said DNA sequence wherein an increase or decrease in the amount of RNA transcribed from said DNA sequence is indicative that said candidate agent is a signal transduction modulating agent.

In one embodiment, the modulation is an increase in the activity of the signal transduction pathway.

In another embodiment, the modulation is a decrease in the activity of the signal transduction pathway.

The invention further encompasses a method of identifying an agent that modulates cell growth, the method comprising the steps of: (i) providing a DNA sequence encoding a polypeptide that comprises an RKIP-motif; and (ii) contacting said DNA sequence with a candidate agent, and (iii) measuring the amount of RNA transcribed from said DNA sequence wherein an increase or decrease in the amount of RNA transcribed from said DNA sequence is indicative that said candidate agent is a cell growth modulating agent.

In one embodiment, the modulation is an increase in cell growth.

In another embodiment, the modulation is a decrease in cell growth.

In another embodiment, the modulation occurs in a cell proliferative disease. Preferably the agent causes a decrease in cell growth in a tumor.

The invention further encompasses a method of identifying an agent that modulates apoptosis, said method comprising the steps of: (i) providing a DNA sequence encoding a polypeptide that comprises an RKIP-motif, (ii) contacting said DNA sequence with a candidate agent, and (iii) measuring the amount of RNA transcribed from said DNA sequence wherein an increase or decrease in the amount of RNA transcribed from said DNA sequence is indicative that said candidate agent is an apoptosis modulating agent.

In one embodiment, the modulation is an increase in apoptosis. Preferably the agent causes a modulation in a tumor.

In another embodiment, the modulation is a decrease in apoptosis.

The invention further encompasses a method of identifying an agent that modulates an RKIP-sensitive pathway, the method comprising the steps of: (i) providing a DNA sequence encoding a polypeptide that comprises an RKIP-motif; (ii) contacting said DNA sequence with a candidate agent, and (iii) measuring the amount of RNA transcribed from said DNA sequence wherein an increase or decrease in the amount of RNA transcribed from said DNA sequence is indicative that said candidate agent is a modulator of an RKIP-sensitive pathway.

In one embodiment, the modulation is an increase in the activity of an RKIP-sensitive pathway.

In another embodiment, the modulation is a decrease in the activity of an RKIP-sensitive pathway. Preferably the agent decreases the activity in a tumor cell.

The invention further encompasses a method of identifying an agent that regulates the transcription of a DNA encoding an RKIP motif-containing protein, the method comprising the steps of: i) providing a cell comprising a reporter gene construct wherein expression of the reporter gene is functionally coupled to the transcriptional control region of a DNA encoding an RKIP motif-containing protein ii) measuring the amount of reporter gene expression from the construct in the presence and absence of a candidate agent, wherein an increase or decrease in the expression of the reporter in the presence of the candidate agent is indicative that the candidate agent regulates the transcription of a DNA encoding an RKIP motif-containing protein.

One of skill in the art will understand that any transcriptional control region that is capable of regulating the expression of an RKIP motif-containing protein will be useful in the present invention. The identification of transcriptional control elements is routine in the art (as described in Sambrook et al., “Molecular Cloning: A Laboratory Manual” (New York, Cold Spring Harbor Laboratory, 1989; and Ausubel et al., John Weley & Sons, Inc., Current Protocols in Molecular Biology, 1997) ) Thus, the invention specifically encompasses any transcriptional control region of an RKIP motif-containing protein that is now known or becomes available in the art.”

The invention further encompasses a method of identifying an agent that regulates transcription of a DNA encoding an RKIP motif-containing protein, the method comprising providing a candidate agent and monitoring the mRNA expression levels of the RKIP motif-containing protein.

The invention further encompasses a method of identifying an agent that regulates transcription of a DNA encoding an RKIP motif-containing protein, the method comprising providing a candidate agent and monitoring the modulation of an RKIP-sensitive pathway wherein modulation of the RKIP-sensitive pathway is indicative that the agent regulates transcription of a DNA encoding an RKIP motif-containing protein.

In one embodiment the RKIP-sensitive pathway is a signal transduction pathway.

The invention further encompasses a method of treating a disorder that is associated with inappropriate expression or activity of an RKIP family polypeptide comprising administering an a pharmaceutical composition comprising an agent that regulates the transcription of a DNA encoding an RKIP motif-containing protein to an individual in need of treatment for a cell proliferative disorder.

The invention further encompasses a method of treating a disorder that is associated with inappropriate activity of an RKIP-sensitive signal transduction pathway comprising administering a pharmaceutical composition comprising an agent that regulates the transcription of a DNA encoding an RKIP motif-containing protein to an individual in need of treatment for a disorder that is associated with inappropriate activity of an RKIP-sensitive signal transduction pathway.

The invention further encompasses a method of detecting a condition associated with the activity of an RKIP-sensitive signal transduction pathway comprising: i) measuring the amount of an RKIP motif-encoding RNA present in a tissue sample; and ii) comparing the amount of an RKIP motif-encoding RNA present in the sample to the amount of the RKIP motif-encoding RNA present in a control tissue sample, wherein an increase or decrease in the amount of the RKIP motif-encoding RNA relative to the amount of the RKIP motif-encoding RNA in the control tissue sample is indicative of a condition associated with the activity of an RKIP-sensitive signal transduction pathway.

In one embodiment, the measuring is performed by a method selected from the group consisting of RT-PCR, RNase protection, in situ hybridization, nuclear run-on or runoff, and Northern hybridization.

In one embodiment, the condition is cancer.

The invention further encompasses a method of modulating a signal transduction pathway, comprising providing an agent that regulates the transcription of an RKIP-motif containing protein.

In one embodiment, the modulation is an increase in activity of the signal transduction pathway.

In another embodiment, the modulation is a decrease in activity of the signal transduction pathway.

The invention further encompasses a method of modulating cell growth, comprising providing an agent that regulates the transcription of an RKIP-motif containing protein.

In one embodiment the modulation is an increase in cell growth.

In another embodiment, the modulation occurs in a cell proliferative disease.

In another embodiment, the modulation is a decrease in cell growth.

The invention further encompasses a method of inhibiting the activity of an RKIP-sensitive kinase, comprising providing a cell with an agent that downregulates transcription of an RKIP-motif containing protein.

The invention further encompasses a method of modulating apoptosis comprising the step of providing an agent that regulates the transcription of an RKIP motif-containing protein.

In one embodiment, the modulation is an increase in apoptosis.

In another embodiment, the modulation occurs in a tumor.

In another embodiment, the modulation is a decrease in apoptosis.

Definitions

The term “RKIP motif” means a motif on a polypeptide characterized by the consensus amino acid sequence TLX3DPD(Z)PX3(B)X4EX2H XnYX4PX(2-4)GXHR(O)VX(Z)X3Q wherein the single letter amino acid code is in accordance with the TUB/IUPAC code, X may be any amino acid, Z indicates a hydrophobic amino acid residue, B indicates negatively charged amino acid residue (D or E), O indicates an aromatic amino acid residue (Y or F), and n is an integer from about 10 to about 50. A sequence does not have to be a perfect match with the consensus in order to be an RKIP motif, but must be comprised within a β fold structure composed of two antiparallel β sheets within the molecule. A sequence that is an RKIP motif is preferably at least about 70% similar to the consensus sequence, more preferably about 75% similar, 80% similar, 85% similar, 90% similar, 95% similar, 98% similar or even 100% similar or most preferably, identical to the consensus. Further, the RKIP sequence motif and polypeptides comprising it interact specifically with one or more signal transduction kinases. Amino acid or nucleotide sequence “identity” and “similarity” are determined from an optimal global alignment between the two sequences being compared. An optimal global alignment is achieved using, for example, the Needleman—Wunsch algorithm (Needleman and Wunsch, 1970, J. Mol. Biol. 48:443-453). “Identity” means that an amino acid or nucleotide at a particular position in a first polypeptide or polynucleotide is identical to a corresponding amino acid or nucleotide in a second polypeptide or polynucleotide that is in an optimal global alignment with the first polypeptide or polynucleotide. In contrast to identity, “similarity” encompasses amino acids that are conservative substitutions. A “conservative” substitution is any substitution that has a positive score in the blosum62 substitution matrix (Hentikoff and Hentikoff, 1992, Proc. Natl. Acad. Sci. USA 89: 10915-10919). Typical conservative substitutions are among Ala, Val, Leu and Ile; among Ser and Thr; among the acidic residues Asp and Glu; among Asn and Gln; and among the basic residues Lys and Arg; or aromatic residues Phe and Tyr. In calculating the degree (most often as a percentage) of similarity between two polypeptide sequences, one considers the number of positions at which identity or similarity is observed between corresponding amino acid residues in the two polypeptide sequences in relation to the entire lengths of the two molecules being compared.

The term “RKIP family” means polypeptides or proteins that comprise an RKIP motif as defined herein. In addition to an RKIP motif, all proteins belonging to the RKIP family have 1) a characteristic β fold structure formed by two anti-parallel β sheets, 2) a cavity capable of accepting an anion (preferably a phosphoryl moiety), and 3) the ability to specifically interact (or bind) with one or more signal transduction kinases. The expression or activity of an RKIP family polypeptide is “inappropriate” when the expression or activity is either increased or decreased in a disease or disorder relative to the expression or activity in a normal individual, wherein it is increased or decreased by at least 10%, and preferably by 20%, 35%, 50%, 75%, 90%, 95% or more, up to and including 100%, and, in the case of an increase, up to 5-fold, 10-fold, 20 fold, or even 50-fold or more.

The term “RKIP motif-containing protein” refers to a polypeptide sequence that comprises an RKIP motif as defined herein.

As used herein, “providing a DNA sequence encoding a polypeptide that comprises an RKIP-motif” means providing an isolated cell or nuclei that comprises a DNA sequence encoding a polypeptide that comprises an RKIP-motif or providing an isolated DNA sequence encoding a polypeptide that comprises an RKIP-motif.

As used herein, the term “purified”, “isolated” and like terms relate to the isolation of a cell, a nuclei or a DNA sequence in a form that is substantially free of contaminants normally associated with a cell, nuclei or DNA sequence in a native or natural environment. For example, a “purified” DNA sequence preferably comprises less than 50% (by weight), less than 40%, and more preferably, less than 2% contaminating polypeptides and/or polynucleotides, of an unlike nature from the purified DNA sequence (e.g., with less than 95%-100% sequence identity).

The term “RKIP motif cassette” refers to a nucleic acid sequence consisting essentially of a sequence encoding a polypeptide that is an RKIP motif as defined herein.

As used herein, the term “heterologous amino acid sequence” refers to an amino acid sequence that is not comprised by or drawn from an RKIP family member.

As used herein, the term “fusion protein” refers to a polypeptide comprising linked regions or domains from two or more polypeptides that are not expressed in a linked manner in nature. An “RKIP motif fusion protein” is the sequence encoded by an RKIP motif cassette linked to a heterologous protein domain or domains. An “RKIP motif-containing fusion protein”, in contrast, may include more of the RKIP family protein than the RKIP motif alone, up to and including the entire RKIP family member protein.

As used herein, the term “signal transduction pathway” refers to a system within a cell that transmits information from outside the cell to the cell nucleus, resulting in a change in the expression of one or more genes. Signal transduction pathways most frequently involve the interactions of protein factors that regulate enzymatic activities (e.g., phosphorylation, protease activity) or the association of signal transducing factors with other factors in a cascade of interactions, wherein the cascade serves to amplify and/or direct a signal to a particular set of genes. The term “activity of a signal transduction pathway” refers to both the effect of the signal transduction pathway on the expression of a gene or genes in response to a signal originating outside the cell and to the individual activities (e.g., association or enzyme activity) of the factors that participate in the pathway. Methods of measuring signal transduction pathway activity are described herein or known in the art. The activity of a signal transduction pathway is herein considered increased if it is increased by at least 10%, and preferably at least 20%, 35%, 50%, 75%, 100%, or even 2-fold, 5-fold, 10-fold, 50-fold or more relative to a standard (e.g., normal tissue or cells or tissue or cells not treated with an agent that modulates that pathway). The activity of an RKIP motif-containing polypeptide is considered decreased if an effector function of such polypeptide, as measured by any of the assay methods described herein, is reduced by at least 10%, and preferably at least 20%, 35%, 50%, 75%, 90%, 95%, or even up to and including 100% (i.e., no activity) relative to a standard.

As used herein, “modulates” refers to “increased” or “decreased as defined above.

As used herein, “contacting” refers to mixing in any order.

Methods for measuring the amount of RNA transcribed from a DNA sequence are described herein or known in the art. The “amount of RNA transcribed” from the DNA sequence is considered increased if it is increased by at least 10%, and preferably at least 20%, 35%, 50%, 75%, 100%, or even 2-fold, 5-fold, 10-fold, 50-fold or more relative to a standard (e.g., normal tissue or cells, or tissue or cells not treated with an agent that modulates the pathway (e.g. signal transduction pathway, cell growth, apoptosis, or RKIP sensitive pathway)). amount of RNA transcribed from the DNA sequence is considered decreased, as measured by any of the assay methods described herein, if the amount transcribed is reduced by at least 10%, and preferably at least 20%, 35%, 50%, 75%, 90%, 95%, or even up to and including 100% (i.e., no activity) relative to a standard (e.g., normal tissue or cells, or tissue or cells not treated with an agent that modulates the pathway (e.g. signal transduction pathway, cell growth, apoptosis, or RKIP sensitive pathway)).

As used herein, “downregulates transcription” refers to a reduction in transcription of at least 10%, and preferably at least 20%, 35%, 50%, 75%, 90%, 95% or even up to an including 100% (i.e. no detectable transcription) relative to a standard (e.g., normal tissue or cells, or tissue or cells not treated with an agent that modulates said pathway, cell growth, apoptosis, or RKIP sensitive pathway).

The term “signal transduction kinase” refers to a kinase that is involved in one or more pathways involved in the transmission of signals originating outside the cell to the nucleus. Examples of signal transduction kinases include, but are not limited to Src, Raf-1 (GenBank Accession No. NM_{—}002880), MEK, MEKK, MEKKK, ERK-1, ERK-2, NIK (GenBank Accession No. Y10256), TAK (GenBank Accession No. D76446), MEKK3, A-Raf, B-RAF, IKK-alpha, IKK-beta etc. of a cell for example, a kinase of the Raf/MEK/ERK or NF-κB signal transduction pathways. The “activity” of a signal transduction kinase is defined as the phosphorylation of target proteins. Alternatively, or in addition, “activity” of signal transduction proteins or the signal transduction pathway refers to the biological result of the phosphorylating activity of the kinase, including, for example, cell proliferation, apoptosis, and cell transformation. The activity of a signal transduction pathway may be measured using methods known in the art or described herein, including, for example kinase assays, binding assays (surface plasmon resonance, yeast two-hybrid, FRET, etc.), transcription assays and/or transformation assays. Signal transduction activity is modulated (increased or decreased) if a measurable parameter of signal transduction activity, including, but not limited to, kinase activity, transcription and/or translation of one or more genes or reporter constructs responsive to that signal transduction pathway, or transformation is increased or decreased by at least 10%, and preferably by 20%, 35%, 50%, 75%, 90%, 95% or more, up to and including 100%, and, in the case of an increase, up to 5-fold, 10-fold, 20 fold, or even 50-fold or more.

A “reporter gene construct” refers to a nucleic acid construct comprising a sequence encoding a detectable marker activity that is operatively linked to expression control region(s) that regulate expression of an RKIP motif-bearing polypeptide. Examples of reporter activities include, but are not limited to luciferase, GFP, CAT, β-galactosidase, secreted alkaline phosphatase, and human growth hormone. A “transcriptional control region” refers to a nucleic acid sequence that comprises an element which binds to transcription factor(s) and that mediates the transactivation of a reporter gene in response to that binding. The term “transcriptional control region” can comprise initiation signals, enhancers, and promoters, which induce or control transcription of protein coding sequences to which they are operatively linked. Herein, the expression of the reporter is increased or decreased when the detection of the reporter by a measurable parameter, including but not limited to a quantitative amount, fluorescence, and enzyme activity is increased or decreased by at least 10%, and preferably by 20%, 35%, 50%, 75%, 90%, 95% or more, up to and including 100%, and, in the case of an increase, up to 5-fold, 10-fold, 20 fold, or even 50-fold or more.

As used herein, the term “functionally coupled”, used in reference to a reporter gene construct and a control region means that changes leading to an increase or decrease in the activity of the control region cause a proportional increase or decrease in the expression of the reporter gene.

As used herein, the term “RKIP-sensitive” refers to the property of a protein or a pathway comprising that protein wherein increases or decreases in the expression or activity of an RKIP-motif-containing polypeptide result in a modulation of the activity of that protein or the pathway in which that protein is active. As used herein, the term “RKIP-sensitive phosphorylation” refers to phosphorylation of a polypeptide that is positively or negatively influenced by changes in the expression or activity of one or more RKIP motif-containing polypeptides.

As used herein, the term “condition associated with the activity of an RKIP-sensitive signal transduction pathway” refers to a disease or disorder characterized by the inappropriate activity of a signal transduction pathway that is sensitive to an RKIP motif-containing polypeptide. One may determine whether a pathway is RKIP-sensitive by either overexpressing an RKIP motif-containing polypeptide in cells in which that pathway is active, or by exposing such cells to an agent that modifies and/or mimics the activity of an RKIP motif-containing polypeptide and measuring the activity of the pathway as described herein. An increase or decrease in the activity of the pathway under such conditions is indicative that the pathway is RKIP-sensitive. The activity of a signal transduction pathway is “inappropriate” if the expression of one or more genes regulated by that pathway is increased or decreased in a disease or disorder relative to the expression of such a gene or genes in a normal individual.

As used herein, a “cell proliferative disorder”, synonymous with a “cell proliferative disease” is a disorder characterized by the inappropriate growth or multiplication of one or more cell types relative to the growth of that cell type or types in an individual not suffering from that disease.

The term “agent” means a composition that has the capacity to modify the amount of RNA transcribed from a DNA sequence that encodes a polypeptide that comprises an RKIP-motif. An agent “regulates transcription” of a DNA encoding RKIP motif-containing protein when the amount of RNA transcribed from the DNA sequence is either increased or decreased as described herein.

An “agent” as used herein refers to a compound that either promotes or inhibits the function of the signal transduction pathway, the expression of genes regulated by that pathway, or the ultimate outcome of that pathway's activation (e.g., proliferation, apoptosis, differentiation, etc.). Agents can include any recombinant, modified or natural nucleic acid molecule, library of recombinant, modified or natural nucleic acid molecules, synthetic, modified or natural peptide, library of synthetic, modified or natural peptides; organic or inorganic compound, or library of organic or inorganic compounds (including small molecules) where the agent has the capacity to modify the bioactivity of an RKIP motif-bearing polypeptide.

As used herein, the term “small molecule” refers to compounds having a molecular mass of less than 3000 Daltons, preferably less than 2000 or 1500, still more preferably less than 1000, and most preferably less than 600 Daltons. Preferably but not necessarily, a small molecule is not an oligopeptide.

The term “cell” as used herein means the smallest structural unit of an eukaryotic organism that is capable of independent functioning, comprising one or more nuclei, cytoplasm, and various organelles that are surrounded by a semi-permeable plasma membrane.

The term “growth” of a cell refers to the proliferative state of a cell as well as to its differentiative state. Accordingly, the term refers to the phase of the cell cycle in which the cell is, e.g., G0, or actively cycling (G1, S, G2, M), as well as to its state of differentiation, e.g., undifferentiated, partially differentiated, or fully differentiated. Without wishing to be limited, differentiation of a cell is usually accompanied by a decrease in the proliferative rate of a cell. As used herein, an increase in cell growth refers to an increase in the rate of proliferation of a cell or population of cells. Cell growth is modulated (increased or decreased) if a measurable parameter of cell growth, including, but not limited to cell number, tissue size, rate of passage through (G1, S, G2, M), or percent of cells in G0, is increased or decreased by at least 10%, and preferably by 20%, 35%, 50%, 75%, 90%, 95% or more, up to and including 100%, and, in the case of an increase, up to 5-fold, 10-fold, 20 fold, or even 50-fold or more.

The process of “apoptosis” is well known, and can be described as a programmed death of cells. As is known, apoptosis is contrasted with “necrosis”, a process when cells die as a result of being killed by a toxic material, or other external effect. Apoptosis is modulated (increased or decreased) if a measurable parameter of apoptosis, including, but not limited to chromatic condensation, membrane blebbing, and DNA fragmentation, is increased or decreased by at least 10%, and preferably by 20%, 35%, 50%, 75%, 90%, 95% or more, up to and including 100%, and, in the case of an increase, up to 5-fold, 10-fold, 20 fold, or even 50-fold or more.

The term “recombinant protein” refers to a polypeptide produced by recombinant DNA techniques, wherein generally, DNA encoding a polypeptide is inserted into a suitable expression vector which is in turn used to transform a host cell to produce the encoded protein. Moreover, the phrase “derived from”, with respect to a recombinant gene, is meant to include within the meaning of “recombinant protein” those proteins having an amino acid sequence of a native polypeptide, or an amino acid sequence generated by mutations including substitutions and deletions (including truncation) and/or additions to a polypeptide sequence as it occurs in nature.

As used herein, “stringent conditions” means hybridization will occur only if there is at least 95%, preferably at least 97%, and optimally 100% identity or complementarity between the probe and the sequences it binds. Specific solution compositions and methods for hybridization under stringent conditions are described herein below.

The term “tissue sample” as used herein means fresh, frozen, or embedded cells, cultured cells, as well as blood and solid tissue samples from a mammal, typically a human. A “control tissue sample” or “standard tissue sample” is a sample taken from either an individual not suffering from a disease or disorder or from an unaffected area of an individual suffering from a disorder. The control or standard is used for comparison with a tissue sample that is being evaluated for a disease or disorder or for the inappropriate expression or activity of an RKIP-sensitive signal transduction pathway.

Herein a “pharmaceutical composition” comprises a therapeutic agent admixed with a physiologically compatible carrier. As used herein, “physiologically compatible carrier” refers to a physiologically acceptable diluent such as water, phosphate buffered saline, or saline, and further may include an adjuvant. Adjuvants such as incomplete Freund's adjuvant, aluminum phosphate, aluminum hydroxide, or alum are materials well known in the art.

### BRIEF DESCRIPTION OF FIGURES

FIG. 1 shows a sequence alignment of amino acid sequences of several RKIP family member proteins. The brackets above the alignment depict the RKIP motif, and the arrows indicate residues shown to be necessary for RKIP activity.

FIG. 2 shows in vitro interaction of RKIP with components of the ERK pathway. a) RKIP interacts with BXB, but not control baits in the yeast two hybrid system. b) Binding of recombinant BXB, full-length Raf-1, MEK-1 and ERK-2 to GST-RKIP beads. “Input”, 1% of the respective proteins used in binding reactions; “GST”, GST-beads. c) Co-immunoprecipitation of RAF-1, MEK and ERK with RKIP in Rat-1 cells. The RKIP antiserum does not precipitate recombinant Raf-1, MEK-1 and ERK-2 proteins individually. d) Co-localization of Raf-1 and RKIP in 208F fibroblasts by confocal microscopy. “Antigen competition”, antisera were pre-absorbed with their cognate antigens.

FIG. 3 shows that inhibition of endogenous RKIP activates AP-1 dependent transcription. a) Microinjection of anti-RKIP antibodies. Quiescent Rat-1 cells were microinjected with the indicated reporter plasmids and antibodies and either left unstimulated or treated with 200 ng/ml TPA or 20 μg/ml forskolin. b) The RKIP antisense vector, pAS-C143, downregulates expression of endogenous RKIP. NIH 3T3 cells were co-transfected with pAS-C143 and a GFP-expressing plasmid. GFP-positive cells were isolated by FACS and immunoblotted with indicated antibodies. c) The activity of an AP-1 reporter gene was measured in serum-starved or TPA-stimulated NIH 3T3 cells following co-transfection with RKIP antisense (pAS-C143) or empty vectors.

FIG. 4 shows that RKIP inhibits Raf- induced AP-1 activation and transformation. a) RKIP reduces basal and BXB-induced AP-1 activity in NIH 3T3 cells co-transfected with a 3xTRE-CAT reporter and the indicated expression plasmids. b) RKIP blocks BXB- but not ERK-induced AP-1 activation. Rat-1 cells were co-microinjected with a 4xTRE-lacZ reporter and the indicated expression vectors. c) RKIP inhibits Raf-dependent proliferation and transformation. NIH 3T3 cells were transfected with BXB, alone or together with RKIP (linked to neo). G418-resistant colonies were counted and scored for morphological transformation. Aliquots of the same transfection were allowed to grow to confluency without drug and were scored for focus formation. A BXB-transformed cell line was infected with LXSH-RKIP retrovirus or LXSH (hygromycin resistant) and seeded in soft agar in the presence of hygromycin. d) RKIP does not inhibit transformation by v-fos, v-src, or mutationally activated MEK in 208F or NIH cells. Data are expressed as reduction in focus formation relative to co-transfection with empty vector (set to 100%).

FIG. 5 shows that RKIP specifically blocks MEK phosphorylation by Raf-1. a) Effect of RKIP on the activation steps of the Raf/MEK/ERK cascade, reconstituted in vitro with purified recombinant proteins. “BSA” 15 μM bovine serum albumin; “Co.” substrate alone; “kn”, kinase negative mutant. b) RKIP does not inhibit activated MEK. HA-MEK-DD or HA-MEK-1 expressed in COS-1 cells were immunoprecipated with anti-HA antibodies from serum starved cells or TPA treated cells, respectively, and assayed for kinase activity. c) RKIP does not inhibit MEK phosphorylation by MEKK-1. ΔMEKK-1 was immunoprecipated from transiently transfected COS-1 cells and used to phosphorylate knMEK. d) RKIP does not inhibit Raf-1 autophosphorylation or phosphorylation of myelin basic protein (MBP).

FIG. 6 shows that RKIP regulates MEK and ERK activation in vivo. a) RKIP downregulation activates MEK. NIH 3T3 cells were co-transfected with GFP and the RKIP antisense plasmid, pAS-C143. GFP positive cells were FACS sorted and immunoblotted with the indicated antisera. b) RKIP antibody microinjection enhances ERK activation. Quiescent NIH 3T3 cells were microinjected with anti-RKIP or control IgG and stimulated with 10 ng/ml TPA for 30 minutes. ERK activation was visualized with a monoclonal anti-phospho-ERK antibody (Sigma) and quantified densitometrically. c) RKIP inhibits MEK-1 activation. COS-1 cells were transiently transfected with HA-MEK and increasing amounts of RKIP expression vectors. Serum starved cells were stimulated with 100 ng/ml TPA for 20 minutes, and the kinase activities of RAF-1 and HA-MEK immunoprecipitates were measured. d) RKIP inhibits stimulation of ERK by v-Ras and v-Src. COS-1 cells were transfected with the indicated expression plasmids plus increasing amounts of RKIP HA-ERK-2 was immunoprecipitated and assay with MBP. e) RKIP inhibits ERK activation by BXB, but not by MEK-DD. COS-1 cells were transfected with the indicated expression vectors and the kinase activity of HA-ERK immunoprecipitates was examined.

FIG. 7 shows that RKIP inhibits the ERK pathway by preventing MEK activation. (a) Rat-cells were microinjected with a TRE-LacZ reporter plasmid and affinity-purified RKIP antibodies or preimmune immunoglobulin G (IgG) and treated as indicated. The MEK Inhibitors PD98059 and U0125 were administered 1 h before microinjection of TPA (110 ng/ml). (b) RKIP antibodies prevent binding of RKIP to Raf-1 or MEK. GST, GST-RKIP, or GST-14-3-3 beads were incubated with saturating amounts of RKIP antibodies (I) or the corresponding preimmune serum (P) and tested for binding of Raf-1 or MEK 1. WB, Western blot. (c) The phosphorylation of kinase-negative MEK-1 (knMEK) by activated Raf-1 was examined in the presence (+) or absence (−) of 10 μM purified RKIP. RKIP was preincubated with RKIP antibodies or the corresponding preimmune serum for 1 h.

FIG. 8 shows that RKIP inhibits Raf-1 by a competitive mechanism. (a) Lineweaver-Burk plot of Raf-1 inhibition by RKIP. Activated GST-Raf-1 was used to phosphorylate GST-MEK-1 in the presence of increasing amounts of RKIP, as indicated. Phosphorylation was quantified with a Fuji phosphorimager. The data shown are the averages of three independent experiments. (b) RKIP disrupts the Raf-1-MEK complexes. GST-MEK and Raf-1 were coexpressed in Sf-9 cells. The GST-MEK-Raf-1 complex was purified by adsorption to glutathione Sepharose beads, washed, and resuspended in PBS. Purified RKIP was added at the concentrations indicated. After 1 h at 4° C., the GST-MEK beads were washed three times with PBS and examined for associated proteins by Western blotting (WB) with the indicated antisera. (c) Raf-1 bound to RKIP does not phosphorylate MEK. A lysate of Sf-9 cells expressing activated Raf-1 was incubated with 5 μg of GST or GST-RKIP beads. Serial dilutions of the same lysate were immunoprecipitated with the anti-Raf serum crafVI. After three washes with PBS, the pellets were resuspended in kinase buffer and incubated with 100 μM ATP and kinase-negative MEK as substrate. MEK phosphorylation was visualized by immunoblotting with a phospho-MEK-specific antiserum. Raf-1 was stained with crafVI.

FIG. 9 shows an analysis of RKIP binding to activated Raf-1, MEK, and ERK. (a) Mitogen activation of Raf-1 decreases its association with RKIP. COS-1 cells were transiently transfected with Raf-1 and RKIP expression vectors. Serum-starved cells were treated with epidermal growth factor (EGF) (20 ng/ml) plus TPA (100 ng/ml) for the times indicated. Raf-1 immunoprecipitates were analyzed for kinase activity, and RKIP immunoprecipitates were examined for Raf-1, IP, Immunoprecipitation, WB, Western blot. (b) Purified RKIP produced in E. coli was tested for binding to GST-Raf and activated (?) GST-Raf beads. GST-Raf proteins were produced in Sf-9 cells and activated by coexpression of RasV12 and Lck. An aliquot of the GST-Raf beads was examined for phosphorylation of kinase-negative MEK (knMEK). (c and d) MEK and ERK proteins were phosphorylated in the presence of [γ-³²P]ATP and tested for binding to GST-RKIP beads. Binding of phosphorylated proteins was detected by autoradiography. Binding of total protein was visualized by Western blotting (WB). The contribution of phosphoproteins to the Western blot signal is minimal, because they represent less than 10% of the total protein.

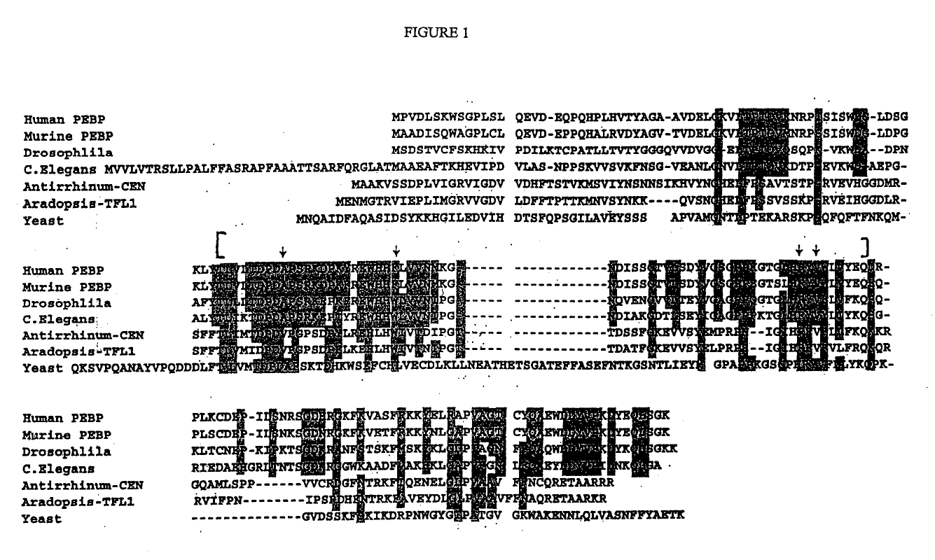

### DETAILED DESCRIPTION

The present invention relates to methods of screening candidate agents for modulation of cell growth, and the use of such agents as pharmaceuticals. More particularly, the invention provides methods by which agents can be screened for their ability to affect transcription of proteins that contain the Raf-1 kinase inhibitor protein (RKIP) motif. The RKIP motif is carried by a family of proteins involved in the regulation of intracellular signal transduction pathways.

The following detailed description discloses how to identify agents that modulate signal transduction pathways by monitoring the transcription of RKIP-motif containing proteins; and methods of using such agents for diagnostic and therapeutic purposes.

A. The RKIP Motif.

The invention is based in part upon the knowledge that RKIP motif containing proteins are involved in the regulation of signal transduction kinases.

1. The RKIP Family

The RKIP motif is a phosphoryl binding pocket comprising the consensus amino acid sequence

TLX₃DPD(Z)PX₃(B)X₄EX₂H_YX₄PX₍₂₋₄₎GXHR(O)VX(Z)X₃Q

wherein the single letter amino acid code is in accordance with the IUB/IUPAC code, Z indicates a hydrophobic amino acid residue, B indicates a negatively charged amino acid residue (D or E), and O indicates an aromatic amino acid residue (Y or F). In members of the RKIP family, this motif is comprised within a structure comprising a characteristic β-fold formed by two antiparallel β-sheets. The characteristic β-fold structure forms a small cavity. Mutagenesis of conserved residues in the cavity or pocket region resulted in loss of the ability of RKIP to interact with Raf-1, loss of Raf-1 inhibitory activity and loss of biological activity in vivo. The pocket region, referred to herein as the “phosphoryl binding pocket” is thus identified as important in the inhibitory function of RKIP and RKIP family members. In addition to effects on the Raf/MEK/ERK pathway, RKIP has been found to inhibit kinases in the NF-κB-mediated signal transduction pathway, including NIK and TAK. The binding and inhibition of kinases in these separate pathways demonstrates that RKIP family members can influence diverse signal transduction pathways.

2. DNA Sequence Encoding a polypeptide comprising an RKIP Motif

The sequence of human RKIP gene is identical to that of the human PEBP gene (GenBank Accession Nos.: S76773, X75252 and X85033 (human); U43206 (mouse); X73137, X75253, X75254, X71873 (monkey). The RKIP motif of the human RKIP gene is encoded by a nucleic acid with the sequence (nucleotides 51-240 of the sequence provided in GenBank ID No. S76773):

5′-ACCTTGGTCCTGACAGACCCGGATGCTCCCAGCAGGAAGGATCCCAA
ATACAGAGAATGGCATCATTTCCTGGTGGTCAACATGAAGGGCAATGACA
TCAGCAGTGGCACAGTCCTCTC CGATTATGTGGGCTCGGGGCCTCCCAA
GGGCACAGGCCTCCACCGCTATGTCTGGCTGGTTTACGAGCAG-3′

A clone encoding an RKIP motif or an RKIP family member protein may be isolated from a cDNA library. Techniques for producing and probing nucleic acid sequence libraries are described, for example, in Sambrook et al., “Molecular Cloning: A Laboratory Manual” (New York, Cold Spring Harbor Laboratory, 1989). In order to isolate a cDNA for human RKIP, one may perform RT-PCR with primers selected from the published PEBP sequence. RKIP clones are also available upon request from the laboratories in which they were cloned (see GenBank listings).

An RKIP family member cDNA can be prepared either by low-stringency probing of a library with a probe derived from the RKIP gene or by probing a cDNA library with a degenerate oligonucleotide (or more correctly, collection of oligonucleotides) designed from the RKIP motif. The preparation and use of degenerate oligonucleotide sequences for the identification of cDNAs is well known in the art, as is low stringency probing with a known cDNA sequence. Alternatively, an expression library, prepared in, for example, λGT11 or another protein display vector system can be probed with an antibody that recognizes an RKIP motif. Antibodies recognizing an RKIP motif may be raised by one of skill in the art using synthetic peptides derived from the consensus sequence as an immunogen.

Once the DNA sequence encoding a polypeptide comprising an RKIP motif is cloned, an isolated nucleic acid sequence comprising the transcriptional control region of the RKIP family member can be prepared by direct synthesis of the nucleic acid sequence, or, alternatively, by PCR using primers that hybridize to the sequences flanking the control region. The isolated sequence can then be cloned into a vector comprising a reporter gene (e.g., β-gal, GFP, luciferase, CAT, etc.) to generate a reporter gene construct. Candidate agents can be screened for their ability to increase or decrease reporter gene expression thereby identifying agents that regulate the transcription of a DNA encoding an RKIP motif-containing protein.

B. Identifying Agents that Regulate Transcription of RKIP Motif-Containing Proteins

In the following discussion, and throughout this specification, it should be understood that methods referring to RKIP and the transcription of RKIP encoding DNA and RNA identified herein apply equally to all members of the RKIP family.

The influence of RKIP over signal transduction events in diverse pathways makes it a strong target for the modulation of those pathways, and thereby, the modulation of the physical manifestations of those pathways. For example, the Raf/MEK/ERK pathway is involved in the regulation of cell proliferation, and the NF-κB transcription factor pathway is involved in the regulation of cell proliferation, apoptosis and immune functions, including but not limited to inflammation. The identification of RKIP and the RKIP family as regulators of kinases involved in these pathways highlights the usefulness of agents that modify the transcription of RKIP family members in the treatment or prevention of diseases or disorders involving these pathways.

Agents that modify the transcription of RKIP family members include those that decrease transcription of RKIP encoding RNA and that increase transcription of RKIP encoding RNA. Agents that increase transcription of RKIP encoding RNA may be used, for example, to inhibit cell proliferation induced by activation of the Raf/MEK/ERK pathways. Such agents are useful in the treatment of cell proliferative disorders, including but not limited to cancer. Alternatively, agents that decrease the transcription of RKIP encoding RNA may be useful, for example, to block inflammation or apoptotic cell death by blocking or modifying the activation of NF-κB. Methods are described below for identifying agents that modify the transcription of RKIP family members. Agents include but are not limited to polypeptides, peptides, nucleic acids, and small molecules.

1. Measuring Transcription

The present invention provides methods for identifying agents that modulate RKIP sensitive pathways via methods comprising measuring the amount of RNA transcribed from a DNA sequence encoding a polypeptide that comprises an RKIP-motif. The amount of RNA transcribed can be measured by standard methods known in the art. In general, total cellular RNA can be isolated from a biological sample using the single-step guanidinium-thiocyanate-phenol-chloroform method described in Chomczynski and Sacchi, Anal. Biochem. 162:156-159, 1987. Levels of mRNA encoding the RKIP protein are then assayed using any appropriate method, these include, but are not limited to, Northern blot analysis (Harada et al., Cell 63:303-312,1990), S1 nuclease mapping (Fujita et al., Cell 49:357-367,1987), the polymerase chain reaction (PCR), reverse transcription in combination with the polymerase chain reaction (RT-PCR) (Makino et al., Technique 2:295-301,1990), and In Situ Hybridization (Ausubel et al., John Weley & Sons, Inc., Current Protocols in Molecular Biology, 1997).

a. Northern Blot Analysis

For Northern blot analysis, isolated mRNA is separated electrophoretically and contacted with a probe. Briefly RNA samples (10 μg/lane), are separated on formaldehyde-agarose gels and transferred onto Genescreen nylon membrane (NEN-Dupont, Boston, Mass.), as described by Sambrook et al. Molecular cloning: a laboratory manual, 2nd edition, Cold Spring Harbor Laboratory Press, New York, p. 8.46-8.47; 8.60-8.63, 1989. Blots are probed with a fragment of RKIP cDNA radiolabeled, for example, by random hexamer priming (Pharmacia). Blots are initially prehybridized for 4 hours at 42° C. in an appropriate solution, for example, 50% formamide, 4×SSPE, 1% SDS, 0.5% skim milk powder, 10% dextran sulphate and 10 mg/ml sheared salmon sperm DNA. [³² P]-Radiolabeled probe is then added at 10⁶ cpm/ml and the blot further incubated for 16 hours at 42° C. Blots are then washed twice for 10 minutes at room temperature, for example, in 2×SSC, 0.1% SDS, then twice at 65° C. in 0.1×SSC, 0.1% SDS, and then exposed to film. The blots are probed with radiolabeled β-actin cDNA (Clontech), or a suitable control, for example a housekeeping gene such as GAPDH, as an indicator of RNA loading. The amount of hybridization is quantitated to determine relative amounts of expression.

b. RT-PCR

In general, isolated RNA is combined with a primer in a reverse transcriptase (RT) reaction to generate single strand cDNAs ( See Sambrook et al. Molecular cloning: a laboratory manual, 2nd edition, Cold Spring Harbor Laboratory Press, New York, p. 8.46-8.47; 8.60-8.63, 1989). Oligo-dT and RKIP sequence specific oligonucleotides, are employed as a primers in the RT reaction. The single strand cDNAs are then amplified with RKIP sequence specific primers to yield an amplified product. To detect the amplified product, the reaction mixture is typically subjected to agarose gel electrophoresis or another convenient separation technique, and the presence or absence of the RKIP amplified DNA detected by Southern blot analysis ( See Sambrook et al. Molecular cloning: a laboratory manual, 2nd edition, Cold Spring Harbor Laboratory Press, New York, p. 8.46-8.47; 8.60-8.63, 1989).

c. RNAse Protection

S1 nuclease assays are an extremely sensitive method for the detection and quantitation of specific mRNAs and are well known in the art (Fujita et al., Cell 49:357-367,1987). In general, the basis of the assay is solution hybridization of an antisense probe (radiolabeled or nonisotopic) to an RNA sample. After hybridization, single-stranded, unhybridized probe and RNA are degraded by nucleases. The remaining protected fragments are separated on an acrylamide gel and quantitated by autoradiography.

d. Nuclear Runoff Assays

De novo transcription can be measured from isolated nucleic using the Nuclear Runoff Assay, for example as described in Cairo et al., J. Biol. Chem. 269:6405-6409 (1994), Chan et al., Eur. J. Biochem. 220:683-692 (1994) which are hereby incorporated by reference). The assay permits direct measurement and comparison of specific gene transcription in cells under various conditions. Nuclei can be prepared, e.g., by NP-40 cell lysis, by dounce homogenization, or by cell lysis followed by sucrose gradient centrifugation (see, e.g., Sambrook and Ausubel, both supra). Briefly, nascent RNA transcripts can be labeled (such as radiolabeled) and can be used to detect specific RNA transcripts by hybridization to cDNAs immobilized on membranes. Those cDNAs containing complementary sequences are identified by standard methods, such as autoradiography.

The invention further provides for a method of identifying an agent that regulates transcription of a DNA encoding an RKIP motif-containing protein by providing a reporter gene construct that is functionally coupled to the transcriptional control region of a DNA encoding an RKIP motif-containing protein. To do this, an isolated transcriptional control region sequence from a DNA encoding an RKIP motif-containing protein is operatively linked to a reporter gene (e.g., β-gal, GFP, luciferase, CAT, etc.) to generate a reporter construct. The reporter construct is then introduced into a eukaryotic host cell, including for example, insect or mammalian cells, and preferably human cells. The reporter is preferably, but not necessarily, stably integrated into the genome of the host cells. The reporter cells are then treated with candidate agents and the expression of reporter is measured. An increase or decrease in reporter expression in the presence, as compared to the absence, of an agent is indicative of an effect of that agent on transcription of a DNA sequence encoding a polypeptide containing an RKIP motif. It should be noted that reporter assays may also be performed in a cell-free manner using nuclear extracts capable of supporting transcription.

2. Monitoring modulation of an RKIP sensitive pathway

Another aspect of the invention provides a method for identifying an agent that regulates transcription of a DNA encoding an RKIP motif-containing protein comprising providing a candidate agent and monitoring the modulation of an RKIP-sensitive pathway. RKIP is known to modulate signal transduction pathways including those involving Raf/MEK/ERK and NF-κB family members. The modulation of an RKIP-sensitive pathway can be monitored by transcription assays, kinase assays, and transformation assays.

1. Transcription Assays.

RKIP is known to modulate signal transduction pathways including those involving Raf/MEK/ERK and NF-κB family members (e.g., NIK and TAK). These pathways ultimately lead to the modification of the expression of specific genes. Therefore, one may use genes ultimately regulated by these pathways to identify agents that modulate transcription of RKIP family members. To do this, sequences responsive to a given pathway are operatively linked to a reporter gene (e.g., β-gal, GFP, luciferase, CAT, etc.) to generate a reporter construct. The reporter construct is then introduced to eukaryotic host cells, including for example, insect or mammalian cells, and preferably human cells. The reporter is preferably, but not necessarily, stably integrated into the genome of the host cells. These reporter cells are treated with candidate agents and the expression of reporter is measured. An increase or decrease in reporter expression in the presence, as compared to the absence of an agent is indicative of an effect of that agent on the RKIP-modulated pathway. It should be noted that reporter assays may also be performed in a cell-free manner using nuclear extracts capable of supporting transcription.

Examples of transcriptional control elements that are responsive to changes in the expression levels of RKIP include, but are not limited to those responsive to the AP-1 transcription factor and those responsive to NF-κB activity. The consensus AP-1 binding site is the palindrome TGA(C/G)TCA (Lee et al., 1987, Nature 325: 368-372; Lee et al., 1987, Cell 49: 741-752). The AP-1 site is also responsible for mediating induction by tumor promoters such as the phorbol ester 12-O-tetradecanoylphorbol-β-acetate (TPA), and are therefore sometimes also referred to as a TRE, for TPA-response element. AP-1 activates numerous genes that are involved in the early response of cells to growth stimuli. Examples of AP-1-responsive genes include, but are not limited to the genes for Fos and Jun (which proteins themselves make up AP-1 activity), Fos-related antigens (Fra) 1 and 2, IκBα, ornithine decarboxylase, and annexins I and II.

The NF-κB binding element has the consensus sequence GGGGACTTTCC. For a non-limiting listing of NF-κB responsive genes, the control elements of which may be used to make NF-κB responsive reporter constructs, see Table 1. Vectors encoding NF-κB responsive reporters are known in the art or can be readily made by one of skill in the art. Further, NF-κB responsive reporters are commercially available from, for example, CLONTECH.

TABLE 1
TARGET GENES OF NF-κB

| | | |
|---|---|---|
| Gene | Function | Reference |

Cytokines/Chemokines and their modulators

| | | |
|---|---|---|
| CINC | Cytokine-induced | Blackwell et al., |
| | neutrophil | 1994 Ohtsuka |
| | chemoattractant | et al., 1996 |
| *CXCL 11 | Chemokine ligand | Tensen et al., |
| | for CXCR3 | 1999 |
| Eotaxin | β Chemokine, | Hein et al., 1997 |
| | eosinphil- | |
| | specific | |
| Gro a-y | Melanoma growth | Anisowicz et al., |
| | stimulating | 1991 |
| | activity | |
| IFN-γ | Interferon | Sica et al., 1992; |
| | | Sica et al., 1997 |
| IL-1α | Interleukin-1α | Mori and Prager, |
| | | 1996 |
| IL-1β | Interleukin-1β | Hiscott et al., |
| | | 1993 |
| IL-1-receptor | Inhibitor of | Smith et al., |
| antagonist | IL-1 activity | 1994 |
| IL-2 | Interleukin-2 | Serfling et al., |
| | | 1989; Hoyos et |
| | | al., 1989; Lai |
| | | et al., 1995 |
| IL-6 | Interleukin-6, | Libermann and |
| | inflammatory | Baltimore, 1990; |
| | cytokine | Shimizu et al., |
| | | 1990 |
| IL-8 | Interleukin-8, | Kunsch and |
| | α-chemokine | Rosen, 1993 |
| *IL-9 | Interleukin-9 | Zhu et al., 1996a |
| IL-11 | Interleukin-11 | Bitko et al., 1997 |
| IL-12 (p40) | Interleukin-12 | Murphy et al., |
| | | 1995 |
| *IL-15 | Interleukin-15 | Azimi et al., 1998 |
| β-Interferon | Interferon | Hiscott et al., |
| | | 1989; Lenardo |
| | | et al., 1989 |
| IP-10 | α Chemokine | Ohmori and |
| | | Hamilton, 1993 |
| KC | α Chemokine | Ohmori et al., |
| | | 1995 |
| Lymphotoxin α | | Worm et al., |
| | | 1998 |
| Lymphotoxin β | Anchors TNF to | Kuprash et al., |
| | cell surface | 1996 |
| MCP-1/JE | Macrophage chemo- | Ueda et al., 1994 |
| | tactic protein, | |
| | β Chemokine | |
| MIP-1a, β | Macrophage inflam- | Grove and Plumbi, |
| | matory protein-1, | 1993; Widmer et |
| | β Chemokine | al., 1993 |
| MIP-2 | Macrophage inflam- | Widmer et al., |
| | matory protein-1, | 1993 |
| | β Chemokine | |
| RANTES | Regulated upon | Moriuchi et al., |
| | Activation Normal | 1997 |
| | T lymphocyte | |
| | Expressed and | |
| | Secreted, | |
| | β Chemokine | |
| TCA3, T-cell | T-cell activation | Oh and Metcalfe, |
| activation gene 3 | gene 3, | 1994 |
| | β Chemokine | |
| TNFα | Tumor necrosis | Shakhov et al., |
| | factor α | 1990; Collart et |
| | | al., 1990 |
| TNFβ | Tumor necrosis | Paul et al., 1990; |
| | factor β | Messer et al., |
| | | 1990 |

Immunoreceptors

| | | |
|---|---|---|
| B7.1 (CD80) | Co-stimulation | Fong et al., 1996; |
| | of T cells via | Zhao et al., 1996 |
| | CD28 binding | |
| BRL-1 | B-cell homing | Wolf et al., 1998 |
| | receptor | |
| CCR5 | Chemokine receptor | Liu et al., 1998 |
| CD48 | Antigen of | Klaman and Thorley- |
| | stimulated | Lawson, 1995 |
| | lymphocytes | |
| F_{c} epsilon | Receptor for IgE | Richards and Katz, |
| receptor II | | 1997 |
| (CD23) | | |
| IL-2 receptor | IL-2 receptor | Ballard et al., |
| a-chain | subunit | 1988 |
| Immunoglobulin | IgG heavy chaini | Lin and Stavnezer, |
| Cgamma1 | | 1996 |
| Immunoglobulin | IgE heavy chain | Iciek et al., 1997 |
| e heavy chain | | |
| Immunoglobulin | Antibody light | Sen and Baltimore, |
| k light chain | chain | 1986b |
| Invariant Chain I₁ | Antigen presentation | Pessara and Koch, |
| | | 1990 |
| MHC class I (H-2K^{b}) | Mouse histocom- | Israël et al., 1989a; |
| | patibility antigen | Israël et al., 1989b |
| MHC Class I HLA-B7 | Mouse histocom- | (Johnson and Pober, |
| | patibility antigen | 1994) |
| β2 Microglobulin | Binds MHC class I | Israël et al., 1989a; |
| | | Israël et al., 1989b |
| T-cell receptor | T-cell receptor | Jamieson et al., 1989 |
| β chain | subunit | |
| *TNF-Receptor, | High-affinity | Santee and Owen- |
| p75/80 | TNF receptor | Schaub, 1996 |

Proteins involved in antigen presentation

| | | |
|---|---|---|
| Proteasome | Subunit of 26S | Wright et al., 1995 |
| Subunit LMP2 | proteasome, | |
| | cysteine protease | |
| Peptide | Peptide transporter | Wright et al., 1995 |
| Transporter TAP1 | for ER | |

Cell adhesion molecules

| | | |
|---|---|---|
| ELAM-1 | E-selectin, endothelial | Whelan et al., 1991 |
| | cell leukocyte | |
| | adhesion molecule | |
| ICAM-1 | Intracellular | van de Stolpe et |
| | adhesion molecule-1 | al., 1994 |
| MadCAM-1 | Mucosal addressin | Takeuchi and |
| | cell adhesion | Baichwal, 1995 |
| | molecule | |
| P-selectin | Platelet adhesion | Pan and McEver, |
| | receptor | 1995 |
| Tenascin-C | ECM protein controls | Mettouchi et al., |
| | cell attachment and | 1997 |
| | migration, cell | |
| | growth | |
| VCAM-1 | Vascular cell | Iademarco et al., |
| | adhesion molecule | 1992 |

Acute phase proteins

| | | |
|---|---|---|
| Angiotensinogen | Angiotensin precursor, | Brasier et al., |
| | regulates blood | 1990; Ron et al., |
| | pressure | 1990 |
| C4b binding | Complement binding | Moffat and Tack, |
| protein | protein | 1992 |
| Complement | Complement factor | Nonaka and Huang, |
| factor B | | 1990 |
| Complement | Activates extrinsic | Yu et al., 1989 |
| Factor C4 | pathway of | |
| | complement activation | |
| C-reactive | Pentraxin | Zhang et al., 1995 |
| protein | | |
| Lipopolysaccharide | Binds to LPS | Schumann, 1995 |
| binding protein | receptor (CD14) | |
| | with LPS | |
| Pentraxin PTX3 | Pentraxin | Basile et al., 1997 |
| Serum amyloid | Serum component | Edbrooke et al., |
| A precursor | | 1991; Li and Liao, |
| | | 1991 |
| Tissue factor-1 | Activates extrinsic | Mackman et al., 1991 |
| | pathway of | |
| | complement activation | |
| Urokinase-type | Activates fibrinogen | Novak et al., 1991 |
| Plasminogen | for fibrin clot lysis | |
| activator | | |

Stress response genes

| | | |
|---|---|---|
| Angiotensin II | Peptide hormone | Brasier et al., |
| | | 1990 |
| COX-2 | Cyclooxygenase, | Yamamoto et al., |
| | prostaglandin | 1995 |
| | endoperoxide | |
| | synthase | |
| Ferritin H chain | Iron storage | Kwak et al., 1995 |
| | protein | |
| *5-Lipoxygenase | Arachidonic acid | Chopra et al., 1992 |
| | metabolic enzyme, | |
| | leukotriene | |
| | synthesis | |
| 12-Lipoxygenase | Arachidonic acid | Arakawa et al., |
| | metabolic enzyme | 1995 |
| inducible | NO synthesis | Geller et al., 1993 |
| NO-Synthase | | |
| Mn SOD | Superoxide dismutase | Das et al., 1995 |
| NAD(P)H quinone | Bioreductive enzyme | Yao and O'Dwyer, |
| oxidoreductase | | 1995 |
| (DT-diaphorase) | | |
| Phospholipase A2 | Fatty acid | Morri et al., 1994 |
| | metabolism | |

Cell-surface receptors

| | | |
|---|---|---|
| A1 adenosine | Pleiotropic | Nie et al., 1998 |
| receptor | physiological | |
| | effects | |
| Bradykinin | Pleiotropic | Ni et al., 1998 |
| B1-Receptor | physiological | |
| | effects | |
| *CD23 | Cell-surface molecule | Tinnell et al., |
| | | 1998 |
| CD69 | Lectin mainly on | Lopez-Cabrera et |
| | activated T cells | al., 1995 |
| Gal1 Receptor | Galanine receptor, | Lorimer et al., |
| | neuroendocrine | 1997 |
| | peptide | |
| Lox-1 | Receptor for | Nagase et al., 1998 |
| | Oxidized low | |
| | density lipoprotein | |
| Mdr1 | Multiple drug | Zhou and Kuo, 1997 |
| | resistance mediator | |
| | (P-glycoprotein) | |
| Neuropeptide | Pleiotropic | Musso et al., 1997 |
| Y Y1-receptor | physiological | |
| | effects | |
| PAF receptor 1 | Platelet activator | Mutoh et al., 1994 |
| | receptor | |
| RAGE- receptor | Receptor for | Li and Schmidt, |
| for advanced | Advanced Glycation | 1997 |
| glycation end | End products | |
| products | | |

Regulators of apoptosis

| | | |
|---|---|---|
| Bfl1/A1 | Pro-survival Bcl-2 | Grumont et al., |
| | homologue | 1999; |
| | | Zong et al., 1999 |
| Bcl-xL | Pro-survival Bcl-2 | Chen et al., 1999; |
| | homologue | Lee et al., 1999b |
| Nr13 | Pro-survival Bcl-2 | Lee et al., 1999c |
| | homologue | |
| CD95 (Fas) | Pro-apoptotic | Chan et al., 1999 |
| | receptor | |
| Fas-Ligand | Inducer of apoptosis | Matsui et al., 1998 |
| IAPs | Inhibitors of | You et al., 1997; |
| | Apoptosis | Stehlik et al., |
| | | 1998 |
| IEX-1L | Immediate early gene | Wu et al., 1998 |

Growth factors and their modulators

| | | |
|---|---|---|
| G-CSF | Granulocyte Colony | Nishizawa and |
| | Stimulating Factor | Nagata, 1990 |
| GM-CSF | Granulocyte | Schreck and |
| | Macrophage | Baeuerle, 1990 |
| | Colony Stimulating | |
| | Factor | |
| *IGFBP-1 | Insulin-like growth | Lang et al., 1999 |
| | factor binding | |
| | protein-1 | |
| IGFBP-2 | insulin-like growth | Cazals et al., 1999 |
| | factor binding | |
| | protein-2 | |
| M-CSF (CSF-1) | Macrophage Colony | Brach et al., 1991b |
| | Stimulating Factor | |
| PDGF B chain | Platelet-Derived | Khachigian et al., |
| | Growth Factor | 1995 |
| Proenkephalin | Hormone | Rattner et al., |
| | | 1991 |
| *Thrombospondin | Matrix glycoprotein t | Adolph et al., 1997 |
| VEGF C | Vascular Endothelial | Chilov et al., 1997 |
| | Growth Factor | |

Early response genes

| | | |
|---|---|---|
| p22/PRG1 | Rat homology of IEX | Schafer et al., |
| | | 1998 |
| *p62 | Non-proteasomal | Vadlamudi and Shin, |
| | multi-ubiquitin | 1998 |
| | chain binding | |
| | protein | |

Transcription factors

| | | |
|---|---|---|
| A20 | TNF-inducible zinc | Krikos et al., 1992 |
| | finger | |
| c-myb | Proto-oncogene | Toth et al., 1995 |
| c-myc | Proto-oncogene | Duyao et al., 1992 |
| c-rel | Proto-oncogene | Hannink and Temin, |
| | | 1990 |
| IRF-1 | Interferon regula- | Harada et al., 1994 |
| | tory factor-1 | |
| IRF-2 | Interferon regula- | Harada et al., 1994 |
| | tory factor-2 | |
| IkB-a | Inhibitor of | Haskill et al., |
| | Rel/NF-kB | 1991; Sun et al., |
| | | 1993 DeMartin et |
| | | al., 1993 |
| junB | Proto-oncogene | Brown et al., 1995 |
| nfkb2 | NF-kB p100 | Lombardi et al., |
| | precursor | 1995 |
| nfkb1 | NF-kB p105 | Ten et al., 1992 |
| | precursor | |
| p53 | Tumor suppressor | Wu and Lozano, 1994 |

Viruses

| | | |
|---|---|---|
| Adenovirus | Adenovirus | Williams et al., |
| (E3 region) | | 1990 |
| Avian Leukosis | Causes avian | Bowers et al., 1996 |
| Virus | leukosis | |
| Bovine Leukemia | Causes bovine | Brooks et al., 1995 |
| Virus | leukemia | |
| CMV | Cytomegalovirus | Sambucetti et al., |
| | | 1989 |
| EBV (Wp promoter) | Epstein-Barr virus | Sugano et al., 1997 |
| HIV-1 | Human immunodefi- | Nabel and |
| | ciency virus | Baltimore, 1987; |
| | | Griffin et al., |
| | | 1989 |
| HSV | Herpes simplex | Rong et al., 1992 |
| | virus | |
| JC Virus | Polyoma virus | Ranganathan and |
| | | Khalili, 1993 |
| Measles virus | Causes measles | Harcourt et al., |
| | | 1999 |
| SIV | Simian immunodefi- | Bellas et al., 1993 |
| | ciency virus | |
| SV-40 | Simian virus 40 | Kanno et al., 1989 |

Enzymes

| | | |
|---|---|---|
| *Ceramide glycosyl | Glycosphingolipid | Ichikawa et al., |
| transferase | | 1998 |
| Collagenase 1 | Matrix | Vincenti et al., |
| | metalloproteinase | 1998 |
| *Dihydrodiol | Oxidoreductase, | Ciaccio et al., |
| dehydrogenase | oxidation of | 1996 |
| | trans-hydodiols | |
| *GAD67 | Glutamic acid | Szabo et al., 1996 |
| | decarboxylase | |
| Gelatinase B | Matrix | He, 1996 |
| | metalloproteinase | |
| GSTP1-1 | Glutathione | Xia et al., 1996 |
| | transferase | |
| *Glucosel-6- | Hexose | Garcia-Nogales |
| phosphate | monophosphate | et al., 1999 |
| dehydrogenase | | |
| *HO-1 | Hemeoxygenase | Lavrovsky et al., |
| | | 1994 |
| Hyaluronan | Synthesizes | Ohkawa et al., 1999 |
| synthase | hyaluronic acid | |
| Lysozyme | Hydrolyzes bacterial | Phi van, 1996 |
| | cell walls | |
| Mmp-9, matrix | Secreted gelatinase | Bond et al, 1998; |
| metalloproteinaase-9 | involved in metastasis | Farina et al., 1999 |
| *PTGIS, prostaglandin | Prostaglandin | Yokoyama et al., |
| synthase | synthase | 1996 |
| Transglutaminase | Forms isopeptide | Mirza et al., 1997 |
| | bonds | |
| *Xanthine Oxidase | Oxidative metabolism | Xu et al., 1996 |
| | of purines | |

Miscellaneous

| | | |
|---|---|---|
| alpha-1 acid | Serum protein | Mejdoubi et al., |
| glycoprotein | | 1999 |
| Apolipoprotein C III | Apoprotein of HDL | Gruber et al., 1994 |
| *Biglycan | Connective tissue | Ungefroren and |
| | proteoglycan | Krull, 1996 |
| Cyclin D1 | Cell-cycle regulation | Guttridge et al., |
| | | 1999; Hinz et al., |
| | | 1999 |
| *Cyclin D3 | Cell-cycle regulation | Wang et al., 1996b |
| Factor VIII | Hemostasis | Figueiredo and |
| | | Brownlee, 1995 |
| Galectin 3 | β-galactosidase- | Hsu et al., 1996 |
| | binding lectin | |
| HMG14 | High mobility group | Walker and Enrietto, |
| | 14 | 1996 |
| K3 Keratin | Intermediate filament | Wu et al., 1994 |
| | protein | |
| Laminin B2 Chain | Basement membrane | Richardson et al., |
| | protein | 1995 |
| Mts1 | Multiple tumor | Tulchinsky et al., |
| | suppressor | 1997 |
| *Pax8 | Paired box gene | Okladnova et al., |
| | | 1997 |
| *UCP-2 | Uncoupling protein-2 | Lee et al., 1999a |
| Vimentin | Intermediate filament | Lilienbaum et al., |
| | protein | 1990 |
| Wilm's Tumor | Tumor suppressor | Dehbi et al., 1998 |
| Supressor Gene | | |
| α1-antitrypsin | Protease inhibitor | Ray et al., 1995 |

ii. Kinase_Assays.

The phosphorylation of kinase targets may be monitored to detect modulation of an RKIP sensitive pathway. RKIP family members inhibit kinase activity, therefore monitoring the activity of these target kinases in the presence or absence of candidate RKIP transcriptional modulators permits one to determine the effect of a candidate modulator on an RKIP sensitive pathway. A decrease in RKIP target kinase activity is indicative of an increased level of RKIP transcription, while an increase in target kinase activity is indicative of a decreased level of RKIP transcription. In vitro kinase assays are performed essentially as described by Hafner et al. (1994, Mol. Cell. Biol. 14: 6696-6703). Briefly, activated RKIP target kinase (e.g., Raf-1) is incubated under conditions permitting phosphorylation of a target protein or proteins (e.g., kinase-negative His/MEK), where γ-³²P ATP is the source of phosphate, and labeling of the target is measured following immunoprecipitation of kinase target.

iii. Transformation Assays.

RKIP activity reduces the transformation of cells in culture by Raf-1 overexpression. This phenomenon may be used to evaluate compounds or agents that modulate the transcription of RKIP family members by exposing cells transfected with a Raf expression vector to such agents and monitoring for changes in the number of transformed foci or the time required for the generation of foci in the culture. Other indicators of transformation include morphological transformation and anchorage-independent growth. Agents that increase the transcription of RKIP are expected to reduce focus formation, and agents that decrease the transcription of RKIP activity are expected to increase focus formation.

C. Candidate Agents

The candidate modulator or candidate agent may be a synthetic compound, a mixture of compounds, or may be a natural product (e.g. a plant extract or culture supernatant).

Candidate agents from large libraries of synthetic or natural compounds can be screened. Numerous means are currently used for random and directed synthesis of saccharide, peptide, and nucleic acid based compounds. Synthetic compound libraries are commercially available from a number of companies including Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Combinatorial libraries are available and can be prepared. Alternatively, libraries of natural compounds in the form of bacterial, fungal, plant and animal extracts are available from e.g., Pan Laboratories (Bothell, Wash.) or MycoSearch (NC), or are readily produceable by methods well known in the art. Additionally, natural and synthetically produced libraries and compounds are readily modified through conventional chemical, physical, and biochemical means.

Useful compounds may be found within numerous chemical classes. Useful compounds may be organic compounds, or small organic compounds. Small organic compounds have a molecular weight of more than 50 yet less than about 2,500 Daltons, preferably less than about 750, more preferably less than about 350 daltons. Exemplary classes include heterocycles, peptides, saccharides, steroids, and the like. The compounds may be modified to enhance efficacy, stability, pharmaceutical compatibility, and the like. Structural identification of an agent may be used to identify, generate, or screen additional agents. For example, where peptide agents are identified, they may be modified in a variety of ways to enhance their stability, such as using an unnatural amino acid, such as a D-amino acid, particularly D-alanine, by functionalizing the amino or carboxylic terminus, e.g. for the amino group, acylation or alkylation, and for the carboxyl group, esterification or amidification, or the like.

Candidate agents will be effective at varying concentrations, depending on the nature of the agent. Therefore, candidate agents should be screened at varying concentrations. Generally, concentrations from about 10 mM to about 1 fM are preferred for screening. The association constants of agents that modulate transcription of a gene encoding an RKIP family protein and/or bind and/or inhibit RKIP family protein activities will generally in the range of about 1 mM to about 1 fM, and optimally in the range of about 1 μM to about 1 pM or less.

Agents can also be anti-sense nucleic acids. As used herein, antisense therapy refers to administration or in situ generation of oligonucleotide molecules or their derivatives which specifically hybridize (e g., bind) under cellular conditions with the cellular mRNA and/or genomic DNA, thereby inhibiting transcription and/or translation of the subject gene. The binding may be by conventional base pair complementarity, or, for example, in the case of binding to DNA duplexes, through specific interactions in the major groove of the double helix. In general, antisense therapy refers to the range of techniques generally employed in the art, and includes any therapy which relies on specific binding to oligonucleotide sequences.

An antisense construct of the present invention can be delivered, for example, as an expression plasmid which, when transcribed in the cell, produces RNA which is complementary to at least a unique portion of the cellular mRNA. Alternatively, the antisense construct is an oligonucleotide probe which is generated ex vivo and which, when introduced into the cell, causes inhibition of expression by hybridizing with the mRNA and/or genomic sequences of a subject nucleic acid. Such oligonucleotide probes are preferably modified oligonucleotides which are resistant to endogenous nucleases, e.g., exonucleases and/or endonucleases, and are therefore stable in vivo. Exemplary nucleic acid molecules for use as antisense oligonucleotides are phosphoramidate, phosphorothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al. (1988) BioTechniques 6:958-976; and Stein et al. (1988) Cancer Res 48:2659-2668. With respect to antisense DNA, oligodeoxyribonucleotides derived from the translation initiation site, e.g., between the −10 and +10 regions of the nucleotide sequence of interest, are preferred.

Antisense approaches involve the design of oligonucleotides (either DNA or RNA) that are complementary to mRNA. The antisense oligonucleotides will bind to the mRNA transcripts and prevent translation. Absolute complementarity, although preferred, is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the mRNA, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well. (Wagner, R. 1994. Nature 372:333). Therefore, oligonucleotides complementary to either the 5′ or 3′ untranslated, non-coding regions of a gene could be used in an antisense approach to inhibit translation of endogenous mRNA. Oligonucleotides complementary to the 5′ untranslated region of the mRNA should include the complement of the AUG start codon. Antisense oligonucleotides complementary to mRNA coding regions are typically less efficient inhibitors of translation but could also be used in accordance with the invention. Whether designed to hybridize to the 5′, 3′, or coding region of subject mRNA, antisense nucleic acids should be at least six nucleotides in length, and are preferably less that about 100 and more preferably less than about 50, 25, 17 or 10 nucleotides in length.

Regardless of the choice of target sequence, it is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO 88/098 10, published Dec. 15, 1988) or the blood-brain barrier (see, e.g., PCT Publication No. WO 89/10 134, published Apr. 25, 1988), hybridization-triggered cleavage agents (See, e.g., Krol et al., 1988, BioTechniques 6:958-976), or intercalating agents (See, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxytriethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5- oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

The antisense oligonucleotide can also contain a neutral peptide-like backbone. Such molecules are termed peptide nucleic acid (PNA)-oligomers and are described, e.g., in Peny-O'Keefe et al. (1996) Proc. Natl. Acad. Sci. U.S.A. 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methyiphosphonate, an alkyl phosphotriester, and a formacetal or analog thereof.

In yet a further embodiment, the antisense oligonucleotide is an α-anomeric oligonucleotide. An a-anomeric oligonucleotide forms specific double-stranded hybrids with complementary RNA in which, contrary to the usual n-units, the strands run parallel to each other (Gautier et al, 1987, Nucl. Acids Res. 15:6625-6641). The oligonucteotide is a 2′-methylribonucleotide (Inoue et al., 1987, Nucl. Acids Res. 15:6131-12148), or a chimeric RNA-DNA analogue (Jnoue et al., 1987, FEBS Lett. 215:327-330).

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209) and methylphosphonate oligonucleotides can be prepared by use of controlled pore glass polymer supports (Sarini et al., 1988, Proc. Natl. Acad. Sci. U.S.A. 85:7448-7451).

D. Diagnostic and Therapeutic Uses of RKIP Motifs and RKIP Family Proteins

In one embodiment, the present invention provides a method of detecting a condition associated with the activity of an RKIP sensitive signal transduction pathway by measuring the amount of an RKIP motif encoding RNA in a tissue. For any given cell type, there is a normal level of RKIP family expression. Because RKIP family proteins inhibit signal transduction kinases, and because the inappropriate inactivation or activation of signal transduction pathways is associated with diseases or disorders including, but not limited to cancer and immune dysfunction such as autoimmunity, inflammation and immune deficiency, the levels of RKIP family protein mRNA transcripts can be used for diagnosis of diseases or disorders. The determination of the amount of RKIP in a tissue sample is performed by measuring the level of expression of an RKIP motif encoding RNA.

The diagnostic method comprises the steps of obtaining a tissue sample from an individual, contacting a nucleic acid probe that hybridizes under stringent conditions to a RNA encoding an RKIP motif with mRNA of the tissue sample, and determining the amount of hybridization of the probe. An elevation by at least a factor of 2, at least a factor of 5, at least a factor of 20, or at least a factor of 50 or more in the amount of hybridization with the mRNA of the tissue sample as compared to the amount of hybridization with the mRNA of a standard or control sample is an increase according to the invention. Conversely, a reduction by at least 10%, preferably at least 20%, 35%, 50%, 75% or more, up to and including a 100% decrease (i.e., no signal) is a decrease in hybridization signal according to the invention. A control sample is a tissue sample in which the level of RKIP motif-encoding nucleic acid is within the normal range, which is defined herein as the amount of mRNA encoding a given RKIP family member or RKIP motifs in general in a tissue that is not affected by a cell proliferative disorder, plus or minus about 10%.

An increase in transcription of RKIP motif-containing proteins is indicative of a decreased cell proliferative capacity and/or an increased likelihood or susceptibility to apoptosis, inflammation or other phenomena regulated by an RKIP sensitive pathway. In contrast, a decrease in transcription of RKIP motif-containing proteins is indicative of an increased cell proliferative capacity and/or a decreased likelihood or susceptibility to apoptosis, inflammation or other phenomena regulated by an RKIP sensitive pathway. An increase is indicative of a cell proliferative disorder.

In clinical applications, human tissue samples can be screened for RKIP motif encoding RNA as identified herein. Such samples could consist of needle biopsy cores, surgical resection samples, lymph node tissue, or serum. For example, these methods include obtaining a biopsy. In certain embodiments, nucleic acids extracted from these samples may be amplified using techniques well known in the art. The level of detected RKIP encoding RNA is compared with statistically valid groups of control tissue samples.

In one embodiment, the diagnostic method comprises determining whether a subject has an abnormal RKIP mRNA level by Northern blot analysis, reverse transcription-polymerase chain reaction (RT-PCR), in situ hybridization or nuclear runoff assays According to the method, cells are obtained from a subject and the level of RKIP motif encoding mRNA, is determined and compared to the level of RKIP motif encoding mRNA in a control subject or normal tissue from the same subject. An abnormal level of RKIP mRNA is indicative of a condition associated with abnormal expression of RKIP motif-containing polypeptides in the individual.

In one aspect, the method comprises in situ hybridization with a probe derived from an RKIP motif encoding nucleic acid. The method comprises contacting the labeled hybridization probe with a sample of a given type of tissue potentially containing abnormally growing cells as well as normal cells, and determining whether the probe labels particular cells of the given tissue type to a degree significantly different (increased or decreased) than the degree to which it labels other cells of the same tissue type. A significant difference in RKIP-encoding nucleic acid is indicative of a disorder involving an RKIP motif containing protein.

E. Method of Modulating Cell Proliferation According to the Present Invention

In one embodiment, the invention provides a method of modulating cell proliferation by administering to an individual an agent that regulates transcription of an RKIP motif-containing polypeptide. Where one wishes to inhibit cell proliferation, an agonist or agent that increases the transcription of an RKIP motif-containing polypeptide is preferred. In order to determine whether cells that proliferate in a given cell proliferative disorder are or may be RKIP-sensitive, one may use any of the methods disclosed herein that measure RKIP motif-containing polypeptides and/or the levels of nucleic acids encoding such polypeptides. Alternatively, or in addition, cells from a biopsy may be cultured and assayed for sensitivity to an agent that increases transcription of an RKIP containing protein. A cell is sensitive to a transcriptional regulating agent if such an agent results in at least a 20%, and preferably a 35%, 50%, 75%, 100% or 2-fold, 5-fold, 10-fold, 20-fold, 50-fold or greater increase in RKIP motif-containing transcripts relative to the level of transcript detected in the absence of that agent. Alternatively, as would be the case where an agent decreases the transcription of an RKIP motif-containing polypeptide, a cell is sensitive to the agent if such agent results in at least a 10%, 20%, 35%, 50%, 75%, 90%, 95% or even up to and including a 100% decrease in transcript encoding an RKIP motif-containing polypeptide.

In order to modulate cell proliferation, an agent that modulates the transcription of an RKIP motif-containing polypeptide, is administered to an individual in need of such treatment. Treatment is considered successful if, for example, the rate of cell proliferation of a cell proliferative disease (as evidenced by, for example, a slowing of the rate of tumor growth, or even a reduction in the size of a tumor) decreases by at least 20%, preferably at least 35%, 50%, 75%, 90%, 95%, or even up to and including 100%. The same guideline applies where treatment is aimed at, for example, modulating the rate of apoptosis or the degree of inflammation. Alternatively, treatment may be monitored by measuring in biopsies the activity of RKIP-sensitive kinases, the level of gene expression regulated by the RKIP-sensitive pathway, or by measuring the levels of RKIP motif-containing protein or the nucleic acids encoding them in the given tissue. The choice of how to monitor will depend in part upon the nature of the agent. For example, an agent that enhances RKIP kinase inhibiting activity by increasing the number of transcripts and ultimately expression of RKIP may be monitored by monitoring RKIP-sensitive kinase activity in the tissue, while an agent that decreases RKIP expression may be monitored by following the levels of transcript or expression.

F. Method of Modulating Apoptosis According to the Invention

Apoptosis, often referred to as “programmed cell death” or “cell suicide” is a process that has gained attention recently as it has become evident that it plays a role in a number of disease pathologies. Inappropriate programmed cell death has been implicated in, for example, Alzheimer's disease, atherosclerosis, stroke, and dilated cardiomyopathy. In these cases, tissue damage is the result of the inappropriate apoptosis. A failure to undergo apoptosis or to respond to apoptotic stimuli has been implicated in diseases such as cancer and some immune dysfunctions such as inflammatory disorders and autoimmune diseases.

There is evidence, drawn from experiments in which cell lines resistant to apoptosis were rendered sensitive to apoptotic stimuli by the expression of RKIP, that apoptosis is an RKIP-sensitive process (data not shown) Therefore, agents that regulate transcription of RKIP can be useful in inducing apoptosis or rendering cells sensitive to apoptotic stimuli, for example in tumors that are not sensitive to such stimuli. Alternatively, in instances where one wishes to avoid apoptosis, for example in stroke or Alzheimer's disease, an agent that regulates RKIP transcriptional activity can be useful. In order to modulate apoptosis according to the invention, one administers an agent that modulates the transcription of an RKIP motif-containing polypeptide to an individual in need of such treatment. Success may be monitored by, for example, monitoring the size of a tumor, or by monitoring the numbers of apoptotic cells in tissue biopsies. Alternatively, in the case in which one seeks to reduce apoptosis, success may be monitored by biopsies, or by monitoring the progression or regression of disease symptoms. For example, the percent occlusion of major vessels may be monitored to measure success in treatment or prevention of atherosclerosis. If the percent occlusion decreases as defined herein or does not increase, the treatment is successful. In treatment of Alzheimer's, standard indices of a patient's mental status may be used to monitor the success of treatment. An improvement in status during the course of treatment is indicative of successful treatment.

Methods for monitoring apoptosis are well known in the art, and include, for example, enzyme-based assays that detect chromosome fragmentation, electrophoretic assays that detect the same phenomenon (DNA “laddering”), FACS analyses that detect the degree of intercalation of a dye and morphological characterization of cells in tissue samples. A method of modulating apoptosis according to the invention is successful if it results in at least a 20% increase or decrease in apoptosis, depending on the desired effect, and preferably at least a 35%, 50%, 75%, 90%, 95% or even a 100% (or greater, in the case of induction of apoptosis) change in the level of apoptosis after treatment.

G. Methods of Measuring RKIP Motif Containing-Polypeptides

In some aspects of the present invention it may be preferred to determine whether cells that proliferate in a given cell proliferative disorder are or may be RKIP-sensitive. This can be done by determining whether a cell sample obtained from a subject possesses an abnormal amount of RKIP motif, the method comprising: (a) obtaining a cell sample from the subject; (b) quantitatively determining the amount of the marker polypeptide in the sample so obtained; and (c) comparing the amount of the marker polypeptide so determined with a known standard, so as to thereby determine whether the cell sample obtained from the subject possesses an abnormal amount of the marker polypeptide. Such marker polypeptides may be detected by immunohistochemical assays, dot-blot assays, ELISA and the like.

Antibodies can be used to detect the level of polypeptides comprising an RKIP motif. In that embodiment, the method comprises the steps of contacting the test tissue with an antibody specific for an RKIP motif that is expressed at a control or standard level in normal tissue of the same tissue type as the test tissue, and determining the amount of immunocomplex formation. A statistically significant difference in the amount of the immunocomplex formed with the RKIP of a test tissue as compared to a normal tissue of the same tissue type is an indication of abnormal cell growth or increased potential for abnormal cell growth or susceptibility to apoptosis or immune dysfunction. A difference in RKIP protein levels may be either an increase or a decrease; the level is considered decreased if it is at least 10% lower, 20% lower, 35% lower, 50% lower, 75% lower; 90% lower or even as much as 100% lower (i.e., no RKIP proteins) relative to a standard; the level is considered increased if it is at least two fold higher than in the standard, at least 5 fold, 10-fold, 20-fold or even 50-fold or more higher than standard. An increase or a decrease is indicative of a disorder related to RKIP-sensitive signal transduction. Disorders related to RKIP-sensitive signal transduction include, but are not limited to cancer and other cell proliferative diseases, immunodeficiency, autoimmunity, and inflammation.

Another such method includes the steps of: providing an antibody specific for the RKIP motif, the motif being present in cancerous tissue of a given tissue type at a level more or less than the level of the gene product in abnormal tissue of the same tissue type; obtaining from an individual a first sample of tissue of the given tissue type, which sample potentially includes abnormally growing cells; providing a second sample of tissue of the same tissue type (which may be from the same patient or from a normal control, e.g. another individual or cultured cells), this second sample containing normal cells and essentially no abnormal cells; contacting the antibody with protein (which may be partially purified, in lysed but unfractionated cells, or in situ) of the first and second samples under conditions permitting immunocomplex formation between the antibody and the RKIP motif present in the samples; and comparing (a) the amount of immunocomplex formation in the first sample, with (b) the amount of immunocomplex formation in the second sample, wherein a statistically significant difference (increase or decrease) in the amount of immunocomplex formation in the first sample as compared to the amount of immunocomplex formation in the second sample is indicative of the presence of abnormally growing cells in the first sample of tissue.

Immunoassays are commonly used to quantitate the levels of proteins in cell samples, and many immunoassay techniques are known in the art. The invention is not limited to a particular assay procedure, and therefore is intended to include both homogeneous and heterogeneous assay procedures. Exemplary immunoassays which can be conducted according to the invention include fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety, or label group, can be attached to the subject antibodies and is selected so as to meet the needs of various uses of the method which are often dictated by the availability of assay equipment and compatible immunoassay procedures. General techniques to be used in performing the various immunoassays noted above are known to those of ordinary skill in the art.

H. Detecting Modulation of an RKIP Sensitive Pathway

Modulation of an RKIP-sensitive pathway in the presence or absense of a candidate agent and the presence or absence of RKIP is detected by performing any of the following assays described in U.S. Ser. No. 09/265,281, incorporated in its entirety: inhibition of Raf-induced AP-1 activation and transformation; inhibition of Raf-1 phosphorylation by Mek; in vivo regulation of Mek and Erk activation; activation of AP-1 dependent reporter gene by anti-RKIP antibodies. If any of these activities are modulated in the presence of an agent, the agent is tested, as described in Example 1 below, to determine if it modulates transcrition of a DNA encoding an RKIP motif containing protein.

I. Dosage and Administration

The present invention provides for pharmaceutical compositions comprising a therapeutically effective amount of an agent that regulates the transcription of an RKIP motif-containing polypeptide in combination with a pharmaceutically acceptable carrier or excipient. Such carriers include, but are not limited to, saline, buffered saline, dextrose, water, glycerol, ethanol, and combinations thereof. The pharmaceutical compositions may be administered in any effective, convenient manner including, for instance, administration by topical, oral, anal, vaginal, intravenous, intraperitoneal, intramuscular, subcutaneous, intranasal or intradermal routes among others.

In therapy or as a prophylactic, the active agent may be administered to an individual as an injectable composition, for example as a sterile aqueous dispersion, preferably isotonic.

Alternatively the composition may be formulated for topical application for example in the form of ointments, creams, lotions, eye ointments, eye drops, ear drops, mouthwash, and aerosols, and may contain appropriate conventional additives, including, for example, preservatives, solvents to assist drug penetration, and emollients in ointments and creams. Such topical formulations may also contain compatible conventional carriers, for example cream or ointment bases, and ethanol or oleyl alcohol for lotions. Such carriers may constitute from about 1% to about 98% by weight of the formulation; more usually they will constitute up to about 80% by weight of the formulation. Alternative means for systemic administration include transmucosal and transdermal administration using penetrants such as bile salts or fusidic acids or other detergents. In addition, if an agent can be formulated in an enteric or an encapsulated formulation, oral administration may also be possible.

For administration to mammals, and particularly humans, it is expected that the daily dosage level of the active agent will be from 0.01 mg/kg to 10 mg/kg, typically around 1 mg/kg. The physician in any event will determine the actual dosage which will be most suitable for an individual and will vary with the age, weight and response of the particular individual. The above dosages are exemplary of the average case. There can, of course, be individual instances where higher or lower dosage ranges are merited, and such are within the scope of this invention.

### EXAMPLES

### Example 1

Identification of an Agent the Modulates Transcription of a DNA Sequence Encoding a Polypeptide that Comprises an RKIP-motif

An agent that modulates transcription of a DNA sequence encoding a polypeptide that comprises an RKIP motif is identified as follows.

A DNA sequence encoding a polypeptide that comprises an RKIP-motif (for example as described in the section entitled “The RKIP Motif”) is contacted with a candidate agent. Preferably, a DNA sequence encoding a polypeptide that comprises an RKIP motif is present in an isolated population of cells or nuclei and the cells or nuclei are contacted with a candidate agent. Candidate agents of the invention are described in the section entitled, “Identifying Agents that Regulate Transcription of RKIP Motif-Containing Proteins. A candidate agent may be a synthetic compound, a mixture of compounds or a natural product. A candidate agent is mixed with the DNA sequence in a range of concentrations. For primary screening, a useful concentration of a candidate agent according to the invention is from about 1 μM to about 60 μM or more (i.e., 100 μM, 1 mM, 10 mM, 100 mM, 1M etc . . . )

Cells comprising a DNA sequence encoding a polypeptide that comprises an RKIP motif are contacted with a candidate agent for varying amounts of times, for example 30 sec, 5 min, 30 min, 1 hr, 6 hr, 12 hr, 12 hr, 24 hr etc . . . .

RNA is isolated from the cells treated with the agent, as well as from a replicate sample of untreated cells, for example, as described in Chomczynski and Sacchi et al., supra. RNA levels are measured by any of the following methods: Northern blot analysis, RT-PCR, S1 nuclease assay, primer extension analysis. The amount of RNA in the treated and untreated sample is compared. An increase or decrease in the amount of RNA in the treated versus the untreated sample is indicative that the candidate agent is a signal transduction modulating agent.

Alternatively, nuclei are isolated from the treated and untreated cells and nuclear run-on or run-off assays are performed to measure newly transcribed RNA (Cairo et al., J. Biol. Chem. 269:6405-6409 (1994), Chan et al., Eur. J. Biochem. 220:683-692 (1994), and Ausubel et al., eds., Current Protocols in Molecular Biology, Green and Wiley (1992), which are hereby incorporated by reference). This assay permits direct measurement and comparison of specific gene transcription in cells under various conditions. Nuclei can be prepared, e.g., by NP-40 cell lysis, by dounce homogenization, or by cell lysis followed by sucrose gradient centrifugation (see, e.g., Sambrook and Ausubel, both supra). Briefly, nascent RNA transcripts can be labeled (such as radiolabeled) and can be used to detect specific RNA transcripts by hybridization to cDNAs immobilized on membranes. Those cDNAs containing complementary sequences are identified by standard methods, such as autoradiography.

An increase or a decrease in the amount of RNA transcribed from the DNA sequence encoding a polypeptide that comprises an RKIP motif is indicative that an agent modulates transcription of the DNA sequence.

### Example 2

Identification of an Agent the Modulates a Signal Transduction Pathway

An agent that modulates a signal transduction pathway is identified as follows.

A DNA sequence encoding a polypeptide that comprises an RKIP-motif (for example as described in the section entitled “The RKIP Motif”) is contacted with a candidate agent. Preferably, a DNA sequence encoding a polypeptide that comprises an RKIP motif is present in an isolated population of cells or nuclei and the cells or nuclei are contacted with a candidate agent. Candidate agents of the invention are described in the section entitled, “Identifying Agents that Regulate Transcription of RKIP Motif-Containing Proteins. A candidate agent may be a synthetic compound, a mixture of compounds or a natural product. A candidate agent is mixed with the DNA sequence in a range of concentrations. For primary screening, a useful concentration of a candidate agent according to the invention is from about 1 μM to about 60 μM or more (i.e., 100 μM, 1 mM, 10 mM, 100 mM, 1M etc . . . ).

Cells comprising a DNA sequence encoding a polypeptide that comprises an RKIP motif are contacted with a candidate agent for varying amounts of times, for example 30 sec, 5 min, 30 min, 1 hr, 6 hr, 12 hr, 12 hr, 24 hr etc . . . .

RNA or nuclei are isolated as described in Example 1. An increase or decrease in the amount of RNA in the treated versus the untreated sample is indicative that the candidate agent is a signal transduction modulating agent. If analysis of nuclei demonstrate an increase or a decrease in the amount of RNA transcribed from the DNA sequence encoding a polypeptide that comprises an RKIP motif, as compared to nuclei isolated from an untreated sample, that is indicative that an agent modulates transcription of the DNA sequence.

If an agent modulates transcription of the DNA sequence, the agent is assayed for its ability to modulate a signal transduction pathway as described in the sections entitled “Monitoring modulation of an RKIP sensitive pathway” and “Detecting Modulation of an RKIP Sensitive Pathway” and as described in Examples 3-8.

### Example 3

Inhibition of RKIP Activity using Antibodies.

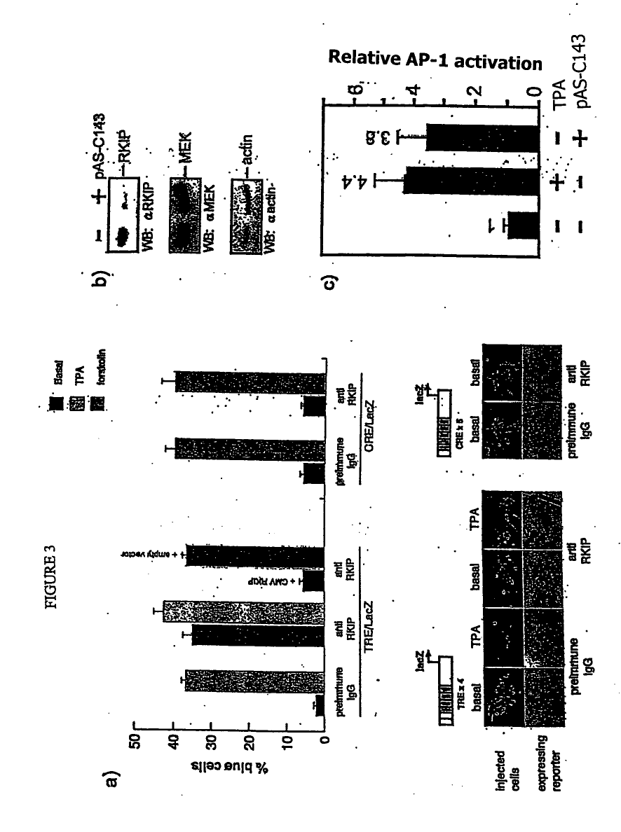

In one embodiment, the RKIP protein inhibiting agent can be an antibody specifically recognizing an RKIP motif. For example, to examine the relevance of the interaction between RKIP and the kinases of the Raf/MEK/ERK module in mammalian cells, endogenous RKIP \was inhibited by antibody microinjection. Since the AP-1 transcription factor is a major target of Raf signaling (Kortenjann et al. Mol. Cell. Biol. 14:4815-4824, 1994;Rapp et al., Oncogene 9:3493-3498, 1994; Kolch et al. Oncogene 8:361-370, 1993), the influence of RKIP on AP-1 activity was tested as shown in FIG. 3.

Microinjections of antibodies and reporter genes were performed as described previously (Lavinsky et al. Proc. Natl. Acad. Sci. USA 95:2920-2925, 1998; Rose et al. J. Cell. Biol. 119:1405-1411, 1992). Briefly, quiescent Rat-1 cells were microinjected with the reporter plasmids and antibodies and either left unstimulated or treated with 200 ng/ml TPA or 20 μg/ml forskolin. The RKIP antiserum was purified over a GST-RKIP affinity column. NIH3T3 cells were stained with an activation specific anti-phospho-ERK monoclonal antibody (Sigma). ERK phosphorylation was quantified by densitometry. For this purpose areas with microinjected cells were randomly photographed, and the staining intensity of whole individual cells was measured using the PcBAS software.

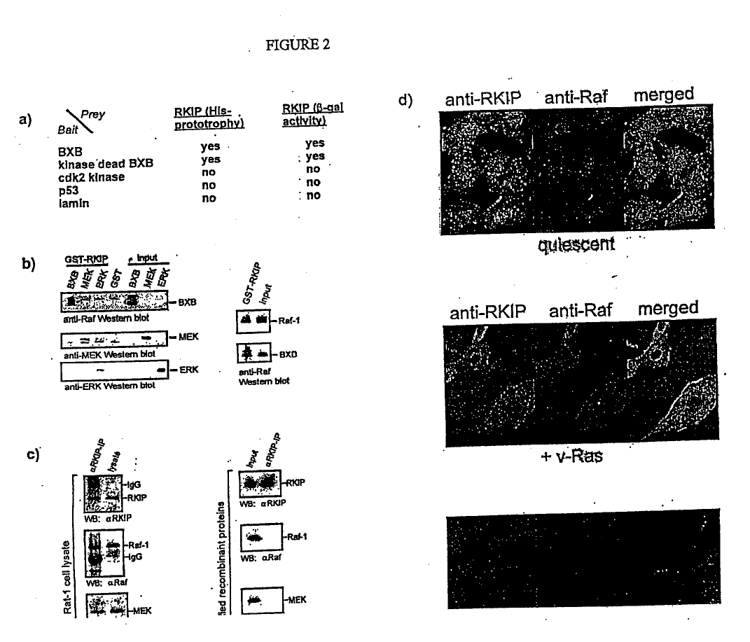

Microinjection of affinity purified anti-RKIP antibodies robustly activated a co-injected AP-1 dependent reporter gene in serum deprived Rat-i fibroblasts as shown in FIG. 3(a). This effect was highly specific, because (i) the injection of control IgG was ineffective; (ii) anti-RKIP IgG did not affect the expression of a cAMP dependent reporter gene; and (iii) co-injection of a RKIP expression vector abolished the AP-1 induction by anti-RKIP IgG. FIG. 2(a) shows that inhibition of endogenous RKIP using anti-RKIP antibodies activates AP-1-dependent transcription.

### Example 4

Inhibition of RKIP Activity using Antisense Molecules.

In one embodiment, the RKIP activity-modulating agent can be an antisense nucleic acid molecule specifically recognizing RKIP motif encoding nucleic acid. For example, we downregulated RKIP protein expression using the RKIP antisense vector, pASC143. The rat RKIP cDNA (Grandy et al. Mol. Endocrinol. 4:1370-1376, 1990) was cloned (i) into pcDNA3 to make p353/RKIP; (ii) into pCMV5 with a triple HA-tag at the N-terminus; and (iii) into pGEX-KG to make GST-RKIP. The pAS-C143 encompasses RKIP nucleotides 1-429 cloned into pCMVori in antisense orientation. pCMVori contains the CMV promoter, polylinker and polyadenylation sequences from pCMV5 inserted into pUCori upstream of the polyoma virus core origin (Gjorup et al. Proc. Natl. Acad. Sci. USA, 91:12125-12129, 1994) 6xIIis-tagged MEK- and GST-fusion proteins were expressed and purified as described (Hafner et al. Mol. Cell. Biol. 14:6696-6703, 1994) RKIP of >95% purity was prepared from GST-RKIP by thrombin cleavage (Guan and Dixon, Anal. Biochemistry 192:262-267, 1991) and subsequent FPLC separation over Superose (Kolch et al., Oncogene, 13:1305-1314, 1996).

The COS-1 cells were transfected as described (Catling et al., 1995, Mol. Cell. Biol. 15: 5214-5225) with 2 μg of HA-ERK-2, BXB, MEK and MEK-DD plasmids and the indicated amounts of p353/RKIP. The total amount of transfected DNA was kept constant using the appropriate vectors as carrier DNA. For RKIP downregulation experiments NIH 3T3 cells were transiently co-transfected using lipofectamine with 0.5 μg of pHACT 20 and 1.5 or 3 μg RKIP antisense expression vector (pAS-C143) or control vector (pCMVori) as indicated. pHACT expresses a truncated polyoma large T construct which has origin binding activity, but does not bind Rb or p53, and boosts the expression of pAS-C143 to high levels. In addition, 0.1 μg of an AP1-Luc reporter was transfected for reporter gene assays. 48 hours post-transfection cells were serum starved for 20 hours and either left untreated or treated with TPA (200 ng/ml) or serum for 5 hours before being collected. Cells were lysed and cell extracts were used for immunoblotting or assayed for luciferase activity. For the GFP sorting experiments 5×10⁶ NIH 3T3 cells were electroporated with either 100 μg pCMVori, 50 μg pCMV-GFP, and 50 μg pHACT or 100 μg pASC143, 50 μg CMV-GFP, and 50 μg CMV-HAC. Two days later cells were trypsinized and sorted for green fluorescent cells by preparative FACS. 100,000 GFP-positive cells were lysed in SDS-gel sample buffer and immunoblotted.

FIG. 3(b) shows that the RKIP antisense vector, pAS-C143, downregulates expression of endogenous RKIP. NIH 3T3 cells were co-transfected with pAS-C 143 and a GFP-expressing plasmid. GFP positive cells were isolated by FACS and immunoblotted with indicated antibodies. FIG. 3(c) shows the measurements of the activity of an AP-1 reporter gene in serum-starved or TPA-stimulated NIH 3T3 cells following co-transfection with RKIP antisense (pAS-C 143) or empty vectors. This vector markedly reduced RKIP protein levels without affecting the expression of MEK-1 or actin as shown in FIG. 3(b). The pAS-C143 substantially induced the AP-1 reporter gene in serum-starved NIH 3T3 cells shown in FIG. 3(c). These data confirm the microinjection results and demonstrate that RKIP suppresses the Raf/MEK/ERK pathway.

### Example 5

Inhibition of Raf Induced AP-1 Activation and Transformation by RKIP Motif.

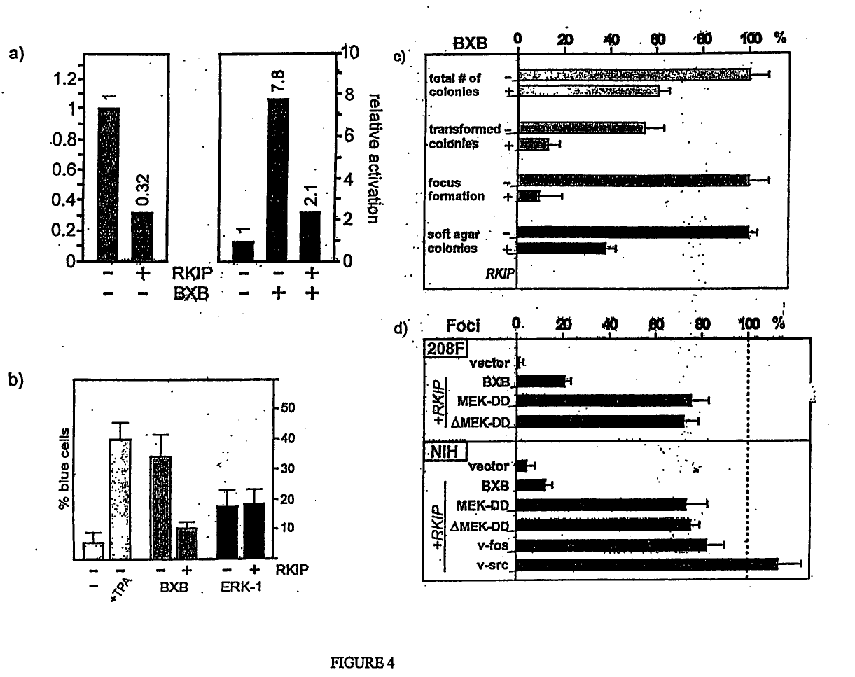

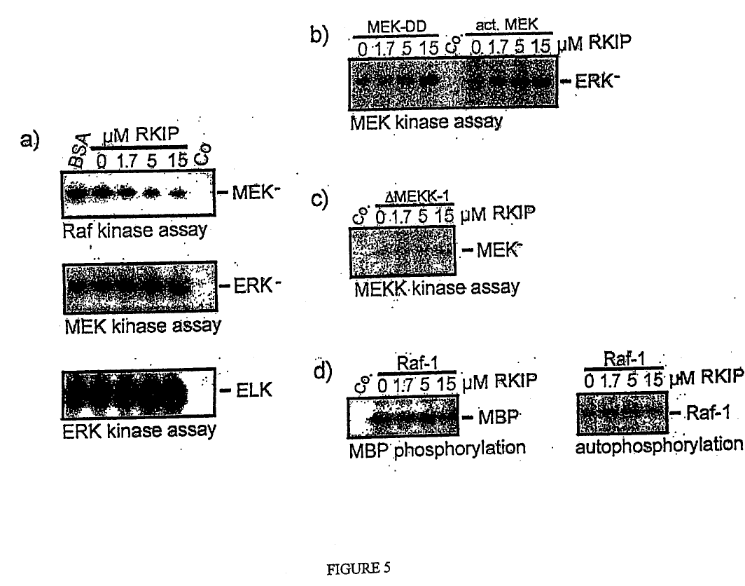

In one embodiment, the present invention provides a method of identifying a cell growth modulating agent by analyzing the effect of a candidate agent on the expression of an RKIP responsive reporter. For example, to show the effect of the RKIP on the activity of the AP-1 mediated transcription, NIH 3T3 cells were co-transfected with a 3xTRE-CAT reporter and the above described expression plasmids. NIH 3T3 and 208F cells were transfected in 6-well plates with 1 μg of pCMV5-BXB and 3 μg of p53/RKIP using Superfect (Qiagen). FIG. 4(a) shows that RKIP reduces basal and BXB-induced AP-1 activity in 3T3 cells. Further, Rat-1 cells were co-microinjected with a 4xTRE-lacZ reporter and the indicated expression vectors and FIG. 4(c) shows that RKIP blocks BXB- but not ERK-induced AP-1 activation.

Additionally, NIH 3T3 cells were transfected with BXB, alone or together with RKIP (linked to neomycin/G418 resistance encoding gene). The G418-resistant colonies were counted and scored for morphological transformation. Aliquots of the same transfection were allowed to grow to confluency without drug and were scored for focus formation. A BXB-transformed cell line was infected with LXSH-RKIP retrovirus or LXSH (hygromycin resistant) and seeded in soft agar in the presence of hygromycin. FIG. 4(c) shows that RKIP inhibits Raf-dependent proliferation and transformation and FIG. 4(d) demonstrates that RKIP does not inhibit transformation by v-fos, v-src, or mutationally activated MEK in 208F or NIH cells. The data are expressed as the reduction in focus formation relative to focus formation upon co-transfection with empty vector (set to 100%).

Overexpression experiments further corroborated this conclusion. RKIP transfection diminished the basal as well as the BXB induced AP-1 activity as shown in FIG. 4(a), and microinjection of an RKIP expression vector impaired AP-1 induction by TPA and BXB as shown in FIG. 4(b). Notably, RKIP did not interfere with AP-1 stimulation by ERK-1.

Next, we tested the effects of RKIP overexpression in transformation assays. In contrast to transient reporter gene assays, transformation assays accommodate the complexity of cellular responses to the chronic deregulation of a single signaling component. RKIP significantly reduced the transformation efficiency of BXB in three distinct assays: morphological transformation, focus formation and anchorage independent growth shown, as in FIG. 4(c). RKIP also decreased total colony yield, albeit to a lesser extent than transformation demonstrating that RKIP interferes with Raf mediated proliferation as well as transformation. In contrast, RKIP impaired the induction of foci by v-fos or mutationally activated MEK alleles only to a small extent and failed to inhibit v-src transformation shown in FIG. 4(d). This indicates that RKIP specifically blocks transformation by the Raf/MEK/ERK pathway and accomplishes this primarily by inhibiting Raf. This is not to say, however, that RKIP or other RKIP family members act only upon this pathway. For example, there is evidence that RKIP inhibits kinases in the NF-κB pathway.

### Example 6

Inhibition of Raf-1 Phosphorylation of MEK by RKIP

To dissect the effects of RKIP on individual activation steps, the Raf/MEK/ERK cascade was reconstructed in vitro using recombinant proteins and analyzing the phosphorylation of the protein components of said cascade.

Kinase assays were done as described in Hafner et al. (Mol. Cell. Biol. 14:6696-6703, 1994). Activated Raf-1 was generated by co-expressing GST-Raf-1 with v-Ras and Lck in Sf-9 cells and collected on glutathione Sepharose beads. Subsequent thrombin cleavage released Raf-1 which was fully active and >90% pure. To activate MEK and ERK in vitro, 20 ng activated Raf-1 was incubated with 40 ng purified His/MEK-1 and 250 ng GST-ERK-2 in Raf kinase buffer containing 20 μM ATP for 20 minutes at 30° C. To measure kinase activities at individual steps the respective downstream components were omitted. The activation reactions were diluted into 50 μl Raf kinase buffer containing 20 μm ATP to yield equimolar concentrations of the kinases to be assayed and incubated with increasing amounts of purified RKIP on ice for 10 minutes. Similar assays may be performed with smaller polypeptides comprising RKIP motifs, or even with a peptide consisting essentially of an RKIP motif as defined herein. Then, 2 μCi [³²P]-γ-ATP and recombinant substrates were added and incubated for 20 minutes at 30° C. As substrates 200 ng kinase negative His/MEK-1 was used for Raf, 1 μg kinase negative GST-ERK for MEK, and 1 μg GST-ELK (New England Biolabs) for ERK. In some assays 1 μg GST-MEK was used as Raf-1 substrate with identical results.

FIG. 5(a) shows the effect of RKIP on the activation steps of the Raf/MEK/ERK cascade reconstituted in vitro with purified recombinant proteins. “BSA” indicates use of 15 μM bovine serum albumin; “Co.” substrate alone; and “kn”, kinase negative mutant. FIG. 5(b) shows that RKIP does not inhibit activated MEK. HA-MEK-DD or HA-MEK-1 expressed in COS-1 cells were immunoprecipitated with anti-HA antibodies from serum starved cells or TPA treated cells, respectively, and assayed for kinase activity. FIG. 5(c) shows that RKIP does not inhibit MEK phosphorylation by MEKK-1. MEKK-1 was immunoprecipitated from transiently transfected COS-1 cells and used to phosphorylate knMEK. Further, FIG. 5(d) indicates that RKIP does not inhibit Raf-1 autophosphorylation or phosphorylation of myelin basic protein (MBP).

RKIP decreased the phosphorylation of MEK by Raf-1, but did not inhibit ERK phosphorylation by MEK or ELK phosphorylation by ERK. In addition, RKIP (i) failed to inhibit MEK-DD, a constitutively active mutant of MEK, or MEK activated by TPA treatment of cells (FIG. 5(b)); (ii) did not prevent MEK phosphorylation by MEKK-1 (FIG. 5(c)); and (iii) did not interfere with Raf-1 autophosphorylation or phosphorylation of MBP by Raf-1 (FIG. 5(d)). These data indicate that RKIP is a very selective inhibitor that specifically blocks MEK activation by Raf. Again, this is not to say that Raf is the only target of RKIP or RKIP family members.

### Example 7

In vivo Regulation of MEK and ERK Activation by RKIP

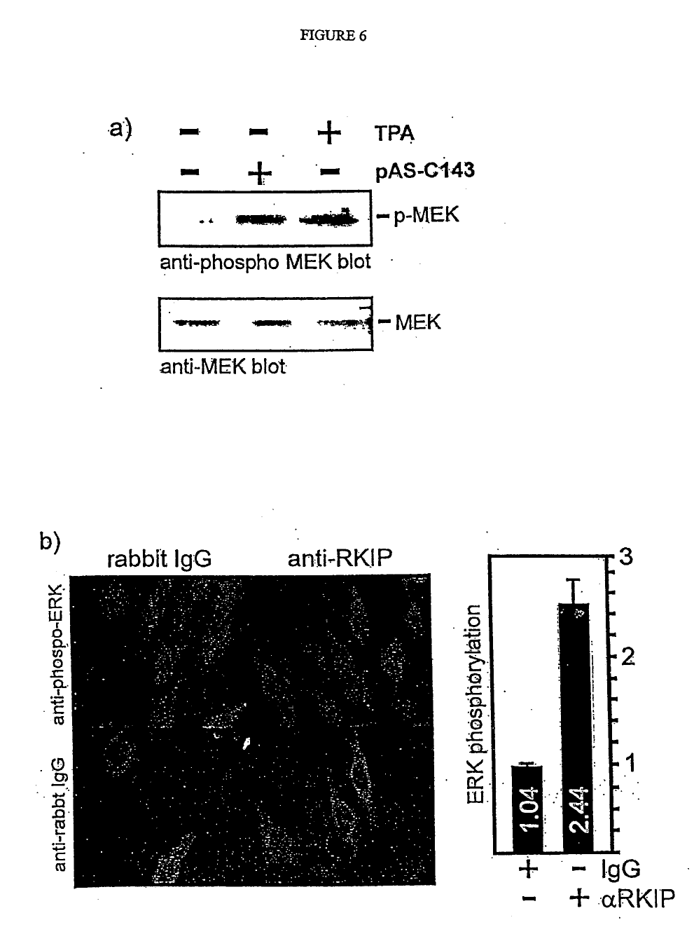

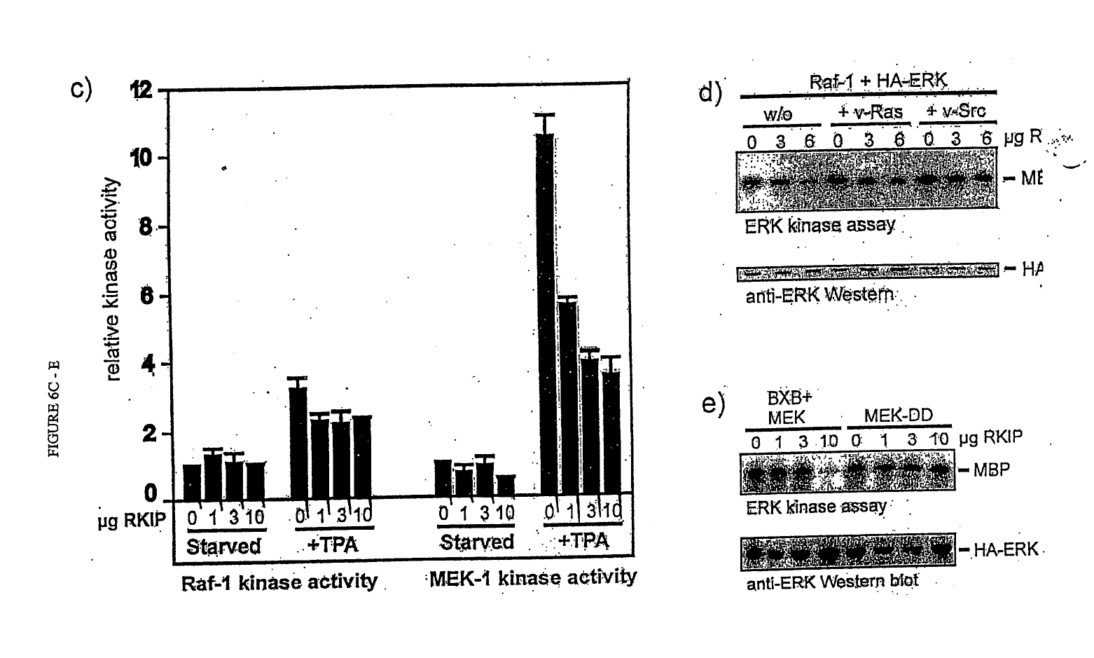

FIG. 6(a) shows that RKIP downregulation activates MEK. NIH 3T3 cells were co-transfected with GFP and the RKIP antisense plasmid, pAS-C143. GFP positive cells were FACS sorted and immunoblotted with the indicated antisera.

FIG. 6(b) demonstrates that RKIP antibody microinjection enhances ERK activation. Here, quiescent NIH 3T3 cells were microinjected with anti-RKIP or control IgG and stimulated with 10 ng/ml TPA for 30 minutes. ERK activation was visualized with a monoclonal anti-phospho-ERK antibody (Sigma) and quantified densitometrically.

FIG. 6(c) shows that RKIP inhibits MEK-1 activation. COS-1 cells were transiently transfected with HA-MEK and increasing amounts of RKIP expression vectors. Serum starved cells were stimulated with 100 ng/ml TPA for 20 minutes, and the kinase activities of Raf-1 and HA-MEK immunoprecipitates were measured.

FIG. 6(d) shows that RKIP inhibits stimulation of ERK by v-Ras and v-Src. COS-1 cells were transfected with the indicated expression plasmids plus increasing amounts of RKIP. HA-ERK-2 was immunoprecipitated and assayed with MBP.

FIG. 6(e) shows that RKIP inhibits ERK activation by BXB, but not by MEK-DD. COS-1 cells were transfected with the indicated expression vectors and the kinase activity of HA-ERK immunoprecipitates was examined.

In vitro, RKIP disrupted the physical interaction between Raf-1 and MEK, which is required for MEK phosphorylation (Kolch et al., 1996, Oncogene 13: 1305-1314), and behaved like a competitive inhibitor for MEK. In vitro binding assays contained 5 μg of GST-fusion protein immobilized on glutathione Sepharose beads and 0.5-5 μg purified recombinant protein in PBS supplemented with 10% bovine serum as nonspecific competitor. Sf-9 cell lysates were used as source of Raf proteins (Hafner et al., 1994, Mol. Cell. Biol. 14: 6696-6703). After incubation for 1 hour at 4° C. the samples were washed 4 times with PBS, resolved by SDS-PAGE and blotted. The blots were developed using ECL (Amersham).

In addition, we investigated whether this mechanism also operated in cells. The downregulation of endogenous RKIP protein expression by the pAS-C 143 antisense vector substantially enhanced MEK phosphorylation on activation specific sites (FIG. 6a). Similarly, microinjection of RKIP antibodies enhanced ERK activation in NIH 3T3 cells (FIG. 6b). In a complementary approach RKIP was over-expressed. Co-transfection of RKIP had only a small influence on the activation of Raf-1 by TPA, but strongly inhibited the activation of MEK in a dose dependent fashion (FIG. 6c). The same results were observed in response to EGF (data not shown). RKIP overexpression also downregulated the activation of ERKs by v-Ras or v-Src oncogenes (FIG. 6d). Co-transfection of increasing amounts of a RKIP expression plasmid inhibited the BXB induced activation of ERK in a dose dependent manner. In contrast, RKIP did not affect ERK activation by MEK-DD (FIG. 6e). These data confirm the in vitro results (FIG. 5), and demonstrate that RKIP regulates the ERK pathway primarily at the Raf/ MEK interface in vivo.

### Example 8

Activation of AP-1 Dependent Reporter Gene by anti-RKIP Antibodies

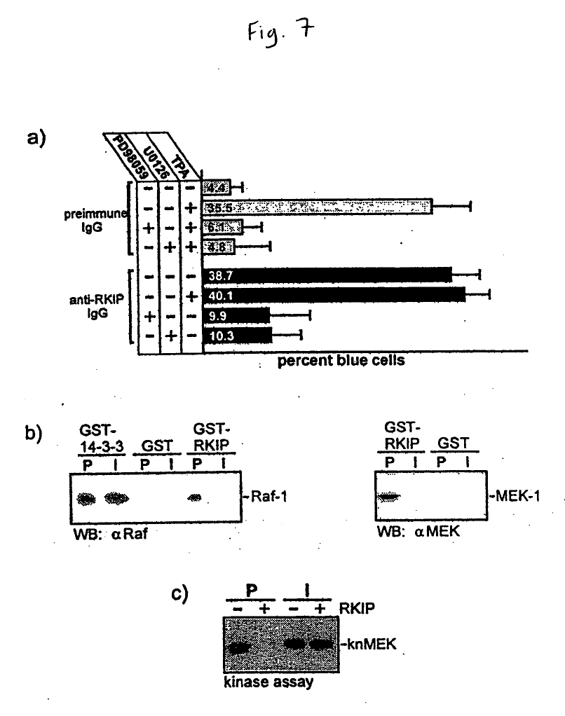

AP-1 luciferase assays and microinjection experiments with affinity-purified RKIP antiserum and TRE-lacZ reporter plasmids were carried out as previously described (Yeung et al. Nature, 401:173-177, 1999). The microinjection of anti-RKIP antibodies raised against the full-length RKIP protein efficiently activated an AP-1 dependent reporter gene. This induction was due to the activation of MEK, since it could be suppressed by two structurally different MEK inhibitors, U0126 and PD98059 (FIG. 7(a)). This showed that the expression of the reporter gene is controlled by the ERK pathway and supports our previous conclusion that RKIP inhibits this pathway by downregulating the activation of MEK by Raf-1 (Yeung et al. Nature, 401:173-177, 1999). The induction of the reporter gene could be completely prevented by co-injection of an RKIP expression vector (Yeung et al. Nature, 401:173-177, 1999), indicating that the RKIP antibodies specifically neutralized RKIP function. These antibodies are therefore useful tools for investigating the molecular mechanism by which RKIP works. The RKIP antiserum interfered with the binding of Raf-1 and MEK to RKIP (FIG. 7(b)). This effect was specific, as (i) the corresponding preimmune serum had no effect (ii) the RKIP antibodies did not prevent the binding of Raf-1 to 14-3-3. Furthermore, the RKIP antibodies reversed the inhibitory effect of RKIP on MEK phosphorylation by Raf-1 (FIG. 7(c)). These results indicated that the inhibitory effect of RKIP on MEK activation by Raf-1 depends on RKIP binding to Raf-1 and/or to MEK.

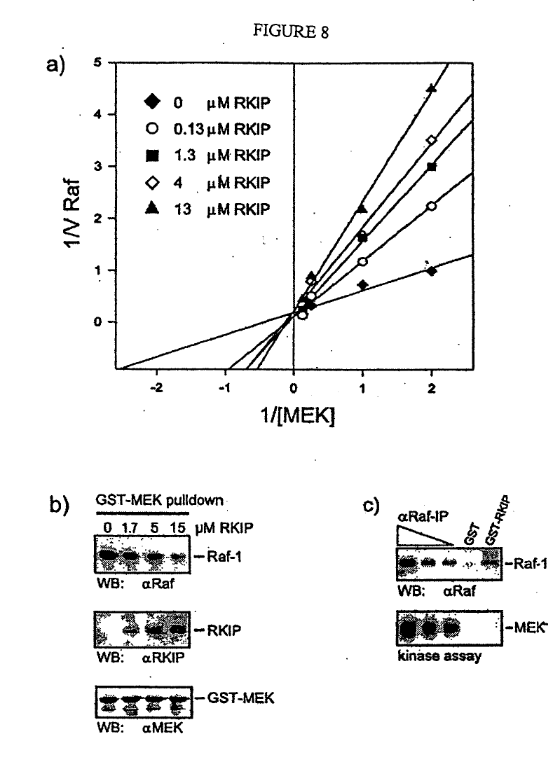

In addition, we studied the nature of RKIP binding. FIG. 8 shows that RKIP inhibits Raf-1 by a competitive mechanism. FIG. 8(a) shows a Lineweaver-Burk plot of Raf-1 inhibition by RKIP. Activated GST-Raf-1 was used to phosphorylate GST-MEK-1 in the presence of increasing amounts of RKIP, as indicated. Phosphorylation was quantified with a Fuji phosphorimager. The data shown are the averages of three independent experiments.

FIG. 8(b) shows that RKIP disrupts the Raf-1-MEK complexes. GST-MEK and Raf-1 were co-expressed in Sf-9 cells. The GST-MEK-Raf-1 complex was purified by adsorption to glutathione Sepharose beads, washed, and resuspended in PBS. Purified RKIP was added at the concentrations indicated. After 1 hour at 4° C., the GST-MEK beads were washed three times with PBS and examined for associated proteins by Western blotting (WB) with the indicated antisera.

FIG. 8(c) demonstrates that Raf-1 bound to RKIP does not phosphorylate MEK. A lysate of Sf-9 cells expressing activated Raf-1 was incubated with 5 μg of GST or GST-RKIP beads. Serial dilutions of the same lysate were immunoprecipitated with the anti-Raf serum crafVI. After three washes with PBS, the pellets were resuspended in kinase buffer and incubated with 100 μM ATP and kinase-negative MEK as substrate. MEK phosphorylation was visualized by immunoblotting with a phospho-MEK-specific antiserum. Raf-1 was stained with crafVI.

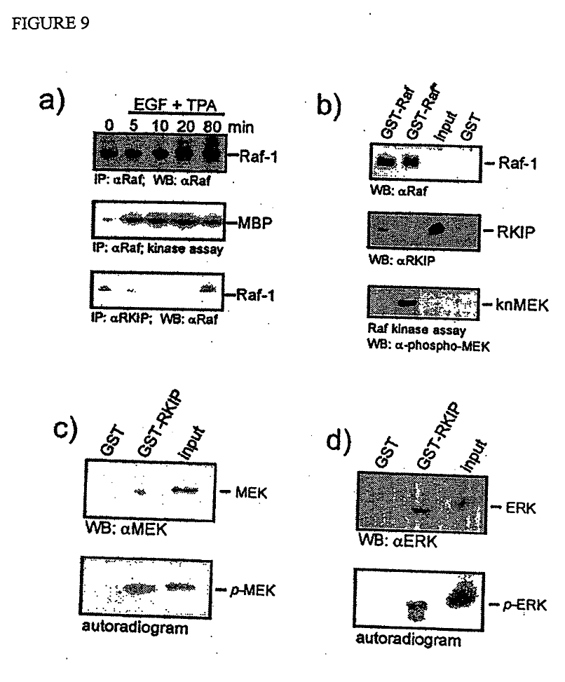

These results also suggested that only the fraction of Raf-1 which is not bound to RKIP is available for activation. Therefore, we examined whether Raf-1 dissociates from RKIP during activation. For this purpose, RKIP and Raf-1 were co-expressed in COS-1 cells shown in FIG. 9(a). Raf-1 co-precipitated with RKIP in quiescent cells. Stimulation of the cells with tetradecanoyl phorbol acetate (TPA) plus epidermal growth factor caused an increase in Raf-1 kinase activity which correlated with a decrease of RKIP association. At later time points, as Raf-1 catalytic activity declined, the levels of Raf-1 co-precipitating with RKIP increased again.

To investigate whether the changes in RKIP association are related to the activation status of Raf-1, the binding of purified RKIP to inactive and activated GST-Raf-1 beads was determined shown in FIG. 9(b). Activated GST-Raf-1 was produced in Sf-9 insect cells co-infected with RasV12 and Lck, which results in a robust activation of the catalytic activity. GST-Raf-1 proteins were purified by adsorption to glutathione Sepharose beads and incubated with recombinant RKIP produced in E. coli. Less RKIP bound to activated GST-Raf-1, indicating that Raf-1 activation weakens the affinity towards RKIP. This finding, however, did not seem to depend on the kinase activity of Raf-1 per se. Kinase-negative Raf-1 mutants, such as RafK375W (Kolch et al., 1991, Nature 349: 426-428) or RafS621A (Morrison et al., 1993, J. Biol. Chem. 268: 17309-17316), as well as activated Raf-1 mutants, such as RafS259D (Morrison et al., 1993, supra) or the isolated kinase domain BXB, bound to RKIP at levels comparable to that of the wild-type Raf-1 (Yeung et al., 1999, supra and data not shown). We also tested whether activation affected the binding of MEK and ERK to RKIP. Purified MEK and ERK were phosphorylated in vitro with recombinant Raf-1 or Raf-1 plus MEK, respectively, and incubated with GST or GST-RKIP beads. The binding reaction products were washed, separated on SDS gels, and immunoblotted with the appropriate antisera. We did not observe any differences in binding between activated and nonactivated forms. However, since only small fractions of MEK and ERK become phosphorylated, we also carried out the phosphorylation in the presence of [γ-³²P] ATP in order to avoid misinterpretation due to low phosphorylation efficiencies shown in FIGS. 9(c) and 9(d). The blots were autoradiographed to detect phosphorylated MEK and ERK and were subsequently stained with the cognate antisera to visualize total protein bound. Under these conditions, binding of phosphorylated MEK and ERK to RKIP was evident.

### Example 9

Identification of an Agent the Modulates Cell Growth

An agent that modulates cell growth is identified as follows.

A DNA sequence encoding a polypeptide that comprises an RKIP-motif (for example as described in the section entitled “The RKIP Motif”) is contacted with a candidate agent. Preferably, a DNA sequence encoding a polypeptide that comprises an RKIP motif is present in an isolated population of cells or nuclei and the cells or nuclei are contacted with a candidate agent. Candidate agents of the invention are described in the section entitled, “Identifying Agents that Regulate Transcription of RKIP Motif-Containing Proteins. A candidate agent may be a synthetic compound, a mixture of compounds or a natural product. A candidate agent is mixed with the DNA sequence in a range of concentrations. For primary screening, a useful concentration of a candidate agent according to the invention is from about 1 μM to about 60 μM or more (i.e., 100 μM, 1 mM, 10 mM, 100 mM, 1M etc . . . ).

Cells comprising a DNA sequence encoding a polypeptide that comprises an RKIP motif are contacted with a candidate agent for varying amounts of times, for example 30 sec, 5 min, 30 min, 1 hr, 6 hr, 12 hr, 12 hr, 24 hr etc . . . .

RNA or nuclei are isolated as described in Example 1. An increase or decrease in the amount of RNA in the treated versus the untreated sample is indicative that the candidate agent is a cell growth modulating agent. If analysis of nuclei demonstrate an increase or a decrease in the amount of RNA transcribed from the DNA sequence encoding a polypeptide that comprises an RKIP motif, as compared to nuclei isolated from an untreated sample, that is indicative that an agent modulates cell growth.

If an agent modulates transcription of the DNA sequence, the agent is assayed for its ability to modulate cell growth as described in the section entitled “Method of modulating cell proliferation according to the present invention”.

### Example 10

Identification of an Agent the Modulates Apoptosis

An agent that modulates apoptosis is identified as follows.

A DNA sequence encoding a polypeptide that comprises an RKIP-motif (for example as described in the section entitled “The RKIP Motif”) is contacted with a candidate agent. Preferably, a DNA sequence encoding a polypeptide that comprises an RKIP motif is present in an isolated population of cells or nuclei and the cells or nuclei are contacted with a candidate agent. Candidate agents of the invention are described in the section entitled, “Identifying Agents that Regulate Transcription of RKIP Motif-Containing Proteins. A candidate agent may be a synthetic compound, a mixture of compounds or a natural product. A candidate agent is mixed with the DNA sequence in a range of concentrations. For primary screening, a useful concentration of a candidate agent according to the invention is from about 1 μM to about 60 μM or more (i.e., 100 μM, 1 mM, 10 mM, 100 mM, 1M etc . . . ).

Cells comprising a DNA sequence encoding a polypeptide that comprises an RKIP motif are contacted with a candidate agent for varying amounts of times, for example 30 sec, 5 min, 30 min, 1 hr, 6 hr, 12 hr, 12 hr, 24 hr etc . . . .

RNA or nuclei are isolated as described in Example 1. An increase or decrease in the amount of RNA in the treated versus the untreated sample is indicative that the candidate agent is an apoptosis modifying agent. If analysis of nuclei demonstrate an increase or a decrease in the amount of RNA transcribed from the DNA sequence encoding a polypeptide that comprises an RKIP motif, as compared to nuclei isolated from an untreated sample, that is indicative that an agent modulates apoptosis.

If an agent modulates transcription of the DNA sequence, the agent is assayed for its ability to modulate apoptosis as described in the section entitled “Method of modulating apoptosis according to the present invention”.

### Example 11

Identification of an Agent that Regulates the Transcription of a DNA encoding an RKIP Motif-containing Protein

An agent that regulates the transcription of a DNA encoding an RKIP motif containing protein is identified as follows. A reporter gene construct comprising a reporter gene (for example luciferase or β-galactosidase) is operationally linked to the transcriptional control region of a DNA encoding an RKIP-motif containing protein. A reporter construct of the invention is prepared using cloning methods well known in the art.

A cell is transfected with the reporter construct and is incubated in the presence or absence of a candidate agent of the invention (as described in Example 1). Expression of the reporter gene is measured by a method that is appropriate for the reporter gene of the construct. An increase or decrease in the expression of the reporter gene in the presence versus absence of a candidate agent is indicative that the agent regulates the transcription of a DNA encoding an RKIP motif-containing protein.

### Example 12

Detecting a Condition Associated with the activity of an RKIP-sensitive Signal Transduction Pathway

A condition associated with the activity of an RKIP-sensitive Signal transduction pathway is identified as follows.

RNA or nuclei are isolated as described in Example 1, from a test tissue sample and a control sample. An increase or decrease in the amount of RNA in the test versus the control sample is indicative of a condition associated with the activity of an RKIP-sensitive signal transduction pathway. If analysis of nuclei from a test sample demonstrate an increase or a decrease in the amount of RNA transcribed from the DNA sequence encoding a polypeptide that comprises an RKIP motif, as compared to nuclei isolated from a control sample, that is indicative of a condition associated with the activity of an RKIP-sensitive signal transduction pathway.

If the above-described RNA analysis is indicative of a condition associated with the activity of an RKIP-sensitive signal transduction pathway, modulation of the activity of an RKIP-sensitive signal transduction pathway is confirmed by measuring RKIP-sensitive signal transduction pathway activity in the test sample and comparing that to the level of activity in a control sample, as described in the sections entitled “Monitoring modulation of an RKIP sensitive pathway” and “Detecting Modulation of an RKIP Sensitive Pathway” and as described in Examples 3-8.

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims.

### OTHER EMBODIMENTS

Other embodiments will be evident to those of skill in the art. It should be understood that the foregoing detailed description is provided for clarity only and is merely exemplary. The spirit and scope of the present invention are not limited to the above examples, but are encompassed by the following claims.

1. A method of identifying an agent that modulates transcription of a DNA sequence encoding a polypeptide that comprises an RKIP-motif comprising the steps of:
(i) providing a DNA sequence encoding a polypeptide that comprises an RKIP-motif; and
(ii) contacting said DNA sequence with a candidate agent; and
(iii) measuring the amount of RNA transcribed from said DNA sequence wherein an increase or decrease in the amount of RNA transcribed from said DNA sequence is indicative that said agent is a transcriptional modulator.

2. A method of identifying an agent that modulates a signal transduction pathway, said method comprising the steps of:
(i) providing a DNA sequence encoding a polypeptide that comprises an RKIP-motif; and
(ii) contacting said DNA sequence with a candidate agent, and
(iii) measuring the amount of RNA transcribed from said DNA sequence wherein an increase or decrease in the amount of RNA transcribed from said DNA sequence is indicative that said candidate agent is a signal transduction modulating agent.

3. The method of claim 2 wherein said modulation is an increase in the activity of said signal transduction pathway.

4. The method of claim 2 wherein said modulation is a decrease in the activity of said signal transduction pathway.

5. A method of identifying an agent that modulates cell growth, said method comprising the steps of:
(i) providing a DNA sequence encoding a polypeptide that comprises an RKIP-motif; and
(ii) contacting said DNA sequence with a candidate agent, and
(iii) measuring the amount of RNA transcribed from said DNA sequence wherein an increase or decrease in the amount of RNA transcribed from said DNA sequence is indicative that said candidate agent is a cell growth modulating agent.

6. The method of claim 5 wherein said modulation is an increase in cell growth.

7. The method of claim 5 wherein said modulation is a decrease in cell growth.

8. The method of claim 5 wherein said modulation occurs in a cell proliferative disease.

9. The method of claim 8 wherein said disease is cancer.

10. A method of identifying an agent that modulates apoptosis, said method comprising the steps of:
(i) providing a DNA sequence encoding a polypeptide that comprises an RKIP-motif; and
(ii) contacting said DNA sequence with a candidate agent, and
(iii) measuring the amount of RNA transcribed from said DNA sequence wherein an increase or decrease in the amount of RNA transcribed from said DNA sequence is indicative that said candidate agent is an apoptosis modulating agent.

11. The method of claim 10 wherein said modulation is an increase in apoptosis.

12. The method of claim 11 wherein said modulation occurs in a tumor.

13. The method of claim 10 wherein said modulation is a decrease in apoptosis.

14. A method of identifying an agent that modulates an RKIP-sensitive pathway, said method comprising the steps of:
(i) providing a DNA sequence encoding a polypeptide that comprises an RKIP-motif; and
(ii) contacting said DNA sequence with a candidate agent, and
(iii) measuring the amount of RNA transcribed from said DNA sequence wherein an increase or decrease in the amount of RNA transcribed from said DNA sequence is indicative that said candidate agent is a modulator of an RKIP-sensitive pathway.

15. The method of claim 14 wherein said modulation is an increase the activity of an RKIP-sensitive pathway.

16. The method of claim 14 wherein said modulation occurs in a tumor.

17. The method of claim 14 wherein said modulation is a decrease in the activity of an RKIP-sensitive pathway.

18. A method of identifying an agent that regulates transcription of a DNA encoding an RKIP motif-containing protein, said method comprising providing a candidate agent and monitoring the mRNA expression levels of said RKIP motif-containing protein.

19. A method for treating a disorder associated with inappropriate expression or activity of an RKIP family polypeptide comprising administering a pharmaceutical composition comprising an agent that regulates the transcription of a DNA encoding an RKIP motif-containing protein to an individual in need of treatment of a cell proliferative disorder.

20. A method for treating a disorder associated with inappropriate activity of an RKIP sensitive signal transduction pathway comprising administering a pharmaceutical composition comprising an agent that regulates the transcription of a DNA encoding an RKIP motif-containing protein to an individual in need of treatment for a disorder associated with inappropriate activity of an RKIP sensitive signal transduction pathway.

21. A method of detecting a condition associated with the activity of an RKIP-sensitive signal transduction pathway comprising:
(i) measuring the amount of an RKIP motif-encoding RNA present in a tissue sample; and
(ii) comparing said amount of an RKIP motif-encoding RNA present in said sample to the amount of said RKIP motif-encoding RNA present in a control tissue sample, wherein an increase or decrease in the amount of said RKIP motif-encoding RNA relative to the amount of said RKIP motif-encoding RNA in said control tissue sample is indicative of a condition associated with the activity of an RKIP-sensitive signal transduction pathway.

22. The method of claim 21 wherein said measuring is performed by a method selected from the group consisting of RT-PCR, RNase protection, in situ hybridization, nuclear run-on and Northern hybridization.

23. The method of claim 21 wherein said condition is cancer.

24. A method of modulating a signal transduction pathway, comprising providing an agent that regulates the transcription of an RKIP-motif containing protein.

25. The method of claim 24 wherein said modulation is an increase in activity of said signal transduction pathway.

26 The method of claim 24 wherein said modulation is a decrease in activity of said signal transduction pathway.

27. A method of modulating cell growth, comprising providing an agent that regulates the transcription of an RKIP-motif containing protein.

28 The method of claim 27 wherein said modulation is an increase in cell growth.

29. The method of claim 27 wherein said modulation occurs in a cell proliferative disease.

30. The method of claim 27 wherein said modulation is a decrease in cell growth.

31. A method of inhibiting the activity of an RKIP-sensitive kinase, comprising providing a cell with an agent that downregulates transcription of an RKIP-motif containing protein.

32. A method of modulating apoptosis comprising the step of providing an agent that regulates the transcription of an RKIP motif-containing protein.

33. The method of claim 31 wherein said modulation is an increase in apoptosis.

34. The method of claim 33 wherein said modulation occurs in a tumor.

35. The method of claim 32 wherein said modulation is a decrease in apoptosis.
